(19) United States
(12) Patent Application Publication
(10) Pub. No.: US 2022/0401662 A1
(43) Pub. Date: Dec. 22, 2022

(54) ACOUSTIC NEBULISER FOR DELIVERY OF ACTIVE AGENTS

(71) Applicant: Royal Melbourne Institute of Technology, Melbourne, Victoria (AU)

(72) Inventors: Leslie YEO, Melbourne (AU); Amgad REZK, Melbourne (AU); Amarin MCDONNELL, Melbourne (AU); Callum KNIGHT, Melbourne (AU)

(73) Assignee: Royal Melbourne Institute of Technology, Melbourne, Victoria (AU)

(21) Appl. No.: 17/766,446

(22) PCT Filed: Oct. 5, 2020
(86) PCT No.: PCT/AU2020/051072

§ 371 Date: Apr. 4, 2022

(51) Int. Cl. A61M 11/00; B05B 17/06

(52) U.S. Cl. CPC A61M 11/005; B05B 17/0676; A61M 2205/0216; A61M 2205/0294; A61M 2205/3389

(57) ABSTRACT

A nebuliser for nebulising liquid droplets, including: a housing; at least one piezoelectric substrate (2) accommodated within the housing and having a transducer surface (2a) upon which is located at least one electroacoustic transducer (48) for generating acoustic wave energy within the at least one piezoelectric substrate (2), and an opposing non-transducer surface (2b); a liquid supply system for supplying a liquid to at least one of the transducer and non-transducer surfaces, the liquid supply system including a reservoir (3) for accommodating the liquid, and at least one relatively rigid supply conduit in contact with the at least one piezoelectric substrate (2) for supplying the liquid from the reservoir (3) to the at least one piezoelectric substrate (2); and a sensor for detecting a volume of liquid on the at least one piezoelectric substrate (2).

### TECHNICAL FIELD

Described embodiments generally directed to nebulisers for nebulising a liquid into small airborne droplets, and in particular to nebulisers using acoustic wave energy to nebulise the liquid.

### BACKGROUND

The use of surface acoustic waves (SAW) for the nebulisation of liquids has been proposed since the 1990′s. See ‘M. Kurosawa et al., ‘Surface acoustic wave atomizer’, Sensors and Actuators A: Physical, 1995, 50, 69-74’. SAW nebulisers have since found application in a variety of fields, including in the administration of active agents. Inhaled medication is the most common form of therapy for asthma, chronic obstructive pulmonary disease (COPD) and for other conditions associated with airflow limitation, such as obstructive bronchitis, emphysema, and cystic fibrosis. There has been extensive research and development in improving the performance of SAW nebulisation platforms in various applications including in fast drop ionization for interfacing with mass spectrometry (see ‘S. R. Heron et al., ‘Surface acoustic wave nebulisation of peptides as a microfluidic interface for mass spectrometry’, Analytical Chemistry, 2010, 82, 3985-3989), nanoparticle synthesis (see ‘J. R. Friend et al, ‘Evaporative self-assembly assisted synthesis of polymeric nanoparticles by surface acoustic wave atomisation’, Nanotechnology, 2008, 19, 1453010), and pulmonary delivery (see A. E. Rajapaksa et al., ‘Effective pulmonary delivery of an aerosolized plasmid DNA vaccine via surface acoustic wave nebulization’, Respiratory Research, 2014, 15, 1).

Despite these continued efforts, the current state-of-the-art has not progressed beyond the research laboratory environment to address issues associated with translating the platform for practical and commercial use. These issues, which are often overlooked by researchers, includes cumbersome and complex fluid chip to reservoir interfacing, weak flow rates, and spurious ejection of large droplets (often constituting a large proportion of the volume delivered), ultimately producing sub-optimal nebulisers that are custom-made to fit a particular laboratory application and can only be run by an expert user rather than a practical and commercially-realisable platform that can be used reliably and easily by end-users.

A particular challenge in using such SAW nebulisation platforms is with regard to issues surrounding the liquid used and their supply to the device. A common approach has been to supply the liquid using a wick placed on a transducer surface of a piezoelectric substrate. An electroacoustic transducer, typically in the form of interdigital transducers (IDTs), is photolithographically applied on the piezoelectric substrate so that the SAW can propagate on the transducer surface. An arrangement using a supply wick is for example shown in U.S. Pat. No. 8,991,722 (Monash University).

The use of a wick on the transducer surface can however lead to undesirable damping of the SAW, heating of the interfacial materials, and sensitivity of the performance depending on the spatial location of the liquid on the device, especially when the acoustic energy is focused on the chip. In addition, a trailing liquid film with a complex multi-step geometry is often present on the device during nebulisation, leading to the production of spurious large drops (>10 μm) and up to 100 μm in size, which are undesirable particularly for pulmonary drug delivery applications where droplets of the order of 1 μm are required for deep lung deposition.

One proposed arrangement to avoid at least some of the above noted issues is shown in International Publication No WO2014/132228 (RMIT University) where the supply wick is brought into contact with a peripheral edge of the piezoelectric substrate to thereby minimise the energy loss associated with the wick and supplied liquid being in contact with the transducer surface. Rather, the interaction of the SAW at the peripheral edge with the supplied liquid leads to the formation of a thin liquid layer from which atomisation can take place.

An alternative approach that has been proposed is to use conventional bulk acoustic waves (BAW) generated within the body of a piezoelectric substrate, rather than SAWs, to nebulise a liquid. U.S. Pat. No. 6,679,436 (Omron) describes a sprayer which uses conventional bulk waves for this purpose. While a SAW platform is used, the SAW is not used to nebulise but to sense the liquid (that is to sense if the liquid is present). Instead, the liquid is applied to the non-transducer surface of the piezoelectric substrate, and the bulk waves generated within the substrate are used to nebulise the liquid.

A problem associated with prior art SAW and BAW platforms is the relatively low nebulisation rates possible with such platforms. SAW platforms typically only have nebulisation rates of about 0.1 ml/min significantly limiting the potential applications of such platforms.

While it is a common belief that nebulisation platforms using SAW are the most efficient wave type, recent research has shown that a combination of both SAW and surface reflected bulk waves (SRBW) have been shown to provide superior liquid nebulisation. (see ‘Amgad R. Rezk et al, ‘Hybrid Resonant Acoustics (HYDRA)’, Advanced Materials, 2016, 1970-1975′). The SRBW is generated when SAW on the transducer surface of the piezoelectric substrate internally reflects between the transducer surface and an opposing non-transducer surface of the substrate located in a parallel adjacent relationship to the substrate surface. The SRBW is therefore generated at the same frequency as the SAW. A hybrid acoustic wave combining both the SAW and SRBW is therefore generated due to their interrelationship, and manifests on both the transducer and non-transducer surfaces. The generation of the SRBW is optimised when the thickness of the substrate is at or around the wavelength of the generated SAW.

International Publication No. WO2016/179664 (RMIT University) describes a nebulisation platform using a hybrid acoustic wave combining SAW and SRBW for nebulising liquids. The liquid may be supplied to a side or end edge of the piezoelectric substrate using a wick or by dipping the substrate edge directly into a reservoir of the liquid. The hybrid acoustic wave (i.e., the SAW and SRBW) then acts to draw a thin film of liquid onto both the IDT surface and the non-IDT surface of the substrate. However, the combined SAW and SRBW nebulisation platform still faces similar concerns as those found in SAW only nebulisation platforms because of the use of a wick in contact with the substrate in one of the described embodiments.

A further challenge for SAW nebuliser systems in administration of active agents, including inhaled medication, is accurate, measurable dosage delivery to ensure the correct dose is received by the patient for therapeutic effect. This prevents a patient from receiving, for example, an overdose. During inhalation, the flow speed of the respiratory gases can be variable which may alter dosage rates or render inhalation therapy less effective, both of which can adversely affect the subject.

Considering the transient change in the load (i.e. piezoelectric chip resistance) with the increase/decrease of the fluid volume on top of a piezoelectric chip, a standard method to detect the presence (ON/OFF state) of a fluid and measure the amount of fluid on the surface of the substrate is via a radio frequency (RF) signal fed into the load/chip. However, this procedure demands connecting the RF signal into an oscilloscope and current probes, which are not only expensive but also very difficult to miniaturise.

Another challenge for SAW nebuliser systems in administration of active agents, including inhaled medication, is preventing loss of atomising liquid from the surface, side or end of the chip. This may occur, for example, where the acoustic waves drive the liquid off the surface prior to atomisation. Loss of atomising liquid from the chip surface may alter dosage rates or render inhalation therapy less effective, which can adversely affect the subject.

These and other SAW nebuliser systems also suffer problems with performance reliability, reproducibility, efficiency and droplet distribution. In particular, systems utilising a single crystal chip are prone to failure due to overheating, pyroelectric failure, and, in some arrangements, require the chip to be in constant contact with a liquid sample. There is scope to improve the performance reliability and efficiency of such devices. Furthermore, achieving appropriate operating parameters, including but not limited to droplet size, geometric standard deviation (GSD) in droplet distribution, stabilization period (i.e. time to use), volumetric atomization rate, and fine particle fraction, for the administration of a diverse range of active pharmaceutical ingredients (APIs) remains a challenge.

The above discussion of background art is included to explain the context of the described embodiments. It is not to be taken as an admission that the background art was known or part of the common general knowledge at the priority date of any one of the claims of the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term, ‘acoustic wave energy’, will be used in the present specification to refer to both travelling and standing surface acoustic waves (SAW), and bulk acoustic waves (BAW) including surface reflected bulk waves (SRBW), and a combination of said waves, in particular, the combined SAW and SRBW.

The term, ‘liquid, will be used in the present specification to refer to pure liquid, or liquid mixtures including functional or therapeutic agents such as pharmaceuticals, plasmid DNA, peptides, perfume and so on.

There is a need for an acoustic nebuliser that addresses one or more of the disadvantages associated with prior art acoustic nebulisers or at least provides an alternative thereto.

### SUMMARY

According to one aspect of the disclosure, there is provided a nebuliser including:

a housing;

at least one piezoelectric substrate accommodated within the housing and having a transducer surface upon which is located at least one electroacoustic transducer for generating acoustic wave energy within the at least one piezoelectric substrate, and an opposing non-transducer surface;

a liquid supply system for supplying a liquid to at least one of the transducer and non-transducer surfaces, the liquid supply system including a reservoir for accommodating the liquid, and at least one relatively rigid supply conduit in contact with the at least one piezoelectric substrate for supplying the liquid from the reservoir to the at least one piezoelectric substrate; and

a sensor for detecting a volume of liquid on the at least one piezoelectric substrate.

In one or more embodiments, the supply conduit may be in the form of a nib or a needle.

According to another aspect of the disclosure, there is provided a nebuliser for nebulising liquid droplets, including:

a housing;

at least one piezoelectric substrate accommodated within the housing and having a transducer surface upon which is located at least one electroacoustic transducer for generating acoustic wave energy within the substrate, and an opposing non-transducer surface;

a compliant material in contact with at least a portion of the perimeter surface of the at least one piezoelectric substrate;

a liquid supply system for supplying a liquid to at least one of the transducer and non-transducer surfaces, the liquid supply system including a reservoir for accommodating the liquid, and at least one supply conduit for supplying the liquid from the reservoir to the substrate; and

a sensor for detecting the volume of liquid on the surface of the substrate.

In one or more embodiments, the compliant material is selected from the group consisting of: adhesive tape, silicone rubber, thermal paste, or combinations thereof. The compliant material may be in contact with at least a portion of a perimeter of a distal end of the at least one piezoelectric substrate.

In one or more embodiments, the at least one supply conduit may be a relatively rigid supply conduit in contact with the at least one piezoelectric substrate.

In one or more embodiments, the at least one supply conduit is selected from the group consisting of a nib, a needle, a wick, a microchannel, or combinations thereof.

In one or more embodiments, the sensor detects the volume of liquid on the surface of the at least one piezoelectric substrate by measuring a change in current across the nebuliser, which may be direct current.

In one or more embodiments, the sensor may be configured to detect a volume of liquid on the transducer surface and/or the non-transducer surface of the at least one piezoelectric substrate.

In one or more embodiments, the nebuliser system (across which the current is measured) includes an electronic circuit and at least one piezoelectric substrate. The electronic circuit may include at least one printed circuit board. In one or more embodiments, the nebuliser may further comprise a control switch responsive to the sensor for controlling operation of the nebuliser.

In one or more embodiments, the nebuliser may be adapted to prevent loss of atomising liquid from the surface, side or end of the substrate. For example, in an embodiment, the nebuliser may further comprise at least one additional and/or opposing electroacoustic transducer for generating acoustic wave energy in an opposing direction to reduce an extent to which liquid is driven off the at least one piezoelectric substrate prior to nebulisation. In still other embodiments, the at least one piezoelectric substrate may further comprise a containing barrier structure for containing and/or preventing loss of liquid applied to the at least one piezoelectric substrate prior to or during nebulisation. In one or more embodiments, the containing barrier structure may comprise a lip, a wall, a gasket, a deposited raised film, and combinations thereof.

In one or more embodiments, the liquid may be gravity fed from the reservoir, or transferred from the reservoir via an active pumping system. In still other embodiments, the liquid supply system further includes a flow regulator for providing a steady flow of liquid therefrom.

In one or more embodiments, the at least one piezoelectric substrate may be supported on a displaceable mount for controlling the contact of the at least one piezoelectric substrate with the supply conduit.

In one or more embodiments, the nebuliser may further include a control means for controlling a size of the nebulised liquid droplets. In one or more embodiments, the control means may include at least one baffle located in a generally parallel and adjacent relationship to at least one of transducer surface or the non-transducer surface.

In one or more embodiments, the housing may further includes an inlet opening, and the reservoir may include a neck portion that can be accommodated within the inlet opening.

In one or more embodiments, the nebuliser may include at least two piezoelectric substrates spaced apart and located in a parallel adjacent relationship.

In one or more embodiments, the droplet size control means may be configured to enable pre-setting of a spacing between the at least two piezoelectric substrates to control a thickness of a meniscus of liquid supplied between adjacent substrate surfaces, to thereby control the size of the nebulised droplets.

In one or more embodiments, the droplet size control means is configured to enable pre-setting of a spacing of the at least two piezoelectric substrates from internal walls of the housing to control a thickness of a meniscus of liquid supplied between the adjacent substrate surfaces and the inner walls, to thereby control the size of nebulised droplets.

In one or more embodiments, the droplet size control means includes a liquid film forming structure in fluid communication with the liquid supply conduit and the at least one piezoelectric substrate to control a thickness of a meniscus of liquid supplied to the at least one piezoelectric substrate to thereby control the size of the nebulised droplets.

In one or more embodiments, the liquid film forming structure includes a web, mesh, one or more fibres, or a slot in the liquid supply conduit or combinations thereof.

In one or more embodiments, at least of portion of the transducer surface, the non-transducer surface, or combinations thereof of the nebuliser of the described embodiments may be patterned.

In one or more embodiments, the generated acoustic wave energy may include surface acoustic waves (SAW) propagated in the transducer surface of the at least one piezoelectric substrate. The acoustic wave energy may include surface reflected bulk waves (SRBW) reflected between the transducer and non-transducer surfaces of the at least one piezoelectric substrate. In an embodiment, the acoustic wave energy may include a combination of surface acoustic waves (SAW) propagated in the transducer surface of the at least one piezoelectric substrate and surface reflected bulk waves (SRBW) reflected between the transducer and non-transducer surfaces of the at least one piezoelectric substrate. The surface acoustic waves (SAW) may include standing waves, traveling waves and combinations thereof. The surface reflected bulk waves (SRBW) may include standing waves, traveling waves and combinations thereof. As previously noted, SRBW is generated when SAW on the transducer surface of the piezoelectric substrate internally reflects between the transducer surface and an opposing non-transducer surface of the substrate located in a parallel adjacent relationship to the substrate surface (i.e. the other side of the substrate). The SRBW is therefore generated at the same frequency as the SAW. A hybrid acoustic wave combining both SAW and SRBW may be generated due to their interrelationship, and manifests on both the transducer and opposing non-transducer surfaces.

As noted above, a liquid supply system may supply a liquid to at least one of the transducer and the non-transducer surfaces. In view of this and the fact that acoustic waves may be manifested on both the transducer and opposing non-transducer surfaces, it is appreciated that a liquid sample may be nebulised from the transducer surface, the opposing non-transducer surface, or both the transducer and opposing non-transducer surfaces. In an embodiment, liquid is nebulised from the transducer surface. In another embodiment, liquid is nebulised from the non-transducer surface. In another embodiment, liquid is nebulised from both the transducer and opposing non-transducer surfaces.

The piezoelectric substrate and electroacoustic transducer of described embodiments may also be used to sense a liquid mass on the at least one substrate. Unlike in U.S. Pat. No. 6,679,436 (Omron) where a surface wave, i.e., the SAW, is used for the sensing, a bulk wave, i.e., a BAW generated on the same substrate is used for the sensing in the described embodiments. The electroacoustic transducer for the nebuliser according to the described embodiments may be an interdigital transducer (IDT). The at least one piezoelectric substrate may be formed of Lithium Niobate (LiNbO₃).

In an embodiment, at least a portion of the non-transducer surface may further include a coating comprising at least one metal. In an embodiment, at least a portion of the transducer surface at the distal end of the substrate may further include a coating comprising at least one metal. The at least one metal may be titanium, gold, aluminium, chromium, copper, or combinations thereof.

The piezoelectric substrate may have a thickness at or around a wavelength of the SAW propagated in the transducer surface. This optimises the generation of SRBWs within the substrate.

In one or more embodiments, the liquid is nebulised from the transducer surface, the non-transducer surface, or both the transducer surface and the non-transducer surface.

In the nebuliser according to described embodiments, the liquid may be nebulised to form droplets having a size across a range between 0.1 μm to 100 μm.

In one or more embodiments, the liquid may be nebulised at a nebulisation rate up to 10.0 ml/min.

According to one embodiment of the nebuliser, the housing may be in the form of a cartridge having an external electrical contact connected to the at least one electroacoustic transducer, and an integral liquid supply system.

In one or more embodiments, the at least one piezoelectric substrate is bonded to the displacement mount.

In one or more embodiments, the at least one piezoelectric substrate is bonded to the displacement mount with a sealing that provides a liquid-tight seal between the transducer surface and the displacement mount.

In one or more embodiments, the non-transducer surface comprises one or more electroacoustic transducers.

In one or more embodiments, there is provided a nebuliser for nebulising liquid droplets, including: a housing; at least one piezoelectric substrate accommodated within the housing and having a transducer surface upon which is located at least one electroacoustic transducer for generating acoustic wave energy within the at least one piezoelectric substrate; at least one opposing electroacoustic transducer for generating acoustic wave energy in an opposing direction to reduce an extent to which liquid is driven off the transducer surface prior to nebulisation; and a liquid supply system for supplying a liquid to the at least one piezoelectric substrate.

In one or more embodiments, there is provided a nebuliser for nebulising liquid droplets, including: a housing; at least two piezoelectric substrates accommodated within the housing; each having a respective transducer surface upon which is located at least one electroacoustic transducer for generating acoustic wave energy within the respective piezoelectric substrate; wherein the at least two piezoelectric substrates are spaced apart and located in a parallel adjacent relationship; a liquid supply system for supplying a liquid to at least one of the piezoelectric substrates; and a control means for controlling a size of the nebulised liquid droplets, the control means being configured to enable pre-setting of a spacing between the at least two piezoelectric substrates to control a thickness of a meniscus of liquid supplied between adjacent substrate surfaces, to thereby control the size of the nebulised droplets.

In one or more embodiments, there is provided a nebuliser for nebulising liquid droplets, including: a housing; at least one piezoelectric substrate accommodated within the housing and having a transducer surface upon which is located at least one electroacoustic transducer for generating acoustic wave energy within the substrate, and an opposing non-transducer surface; and a liquid supply system for supplying a liquid to at least one of the transducer and non-transducer surfaces, the liquid supply system including a reservoir for accommodating the liquid, and at least one relatively rigid supply conduit in contact with the at least one piezoelectric substrate for supplying the liquid from the reservoir to the at least one piezoelectric substrate.

In one or more embodiments, there is provided a nebuliser for nebulising liquid droplets, including: a housing; at least one piezoelectric substrate accommodated within the housing and having a transducer surface upon which is located at least one electroacoustic transducer for generating acoustic wave energy within the at least one piezoelectric substrate, and an opposing non-transducer surface; a compliant material in contact with at least a portion of a perimeter surface of the at least one piezoelectric substrate; and a liquid supply system for supplying a liquid to at least one of the transducer and non-transducer surfaces, the liquid supply system including a reservoir for accommodating the liquid, and at least one supply conduit for supplying the liquid from the reservoir to the at least one piezoelectric substrate.

In one or more embodiments, the at least one electroacoustic transducer may be configured to provide an output indicative of a volume of liquid on the at least one piezoelectric substrate.

In one or more embodiments, the output provided by the at least one electroacoustic transducer may be a current.

In one or more embodiments, the nebuliser may further comprise a sensor for detecting a volume of liquid on the at least one piezoelectric substrate.

In one or more embodiments, the at least one electroacoustic transducer may comprise the sensor.

In one or more embodiments, the nebuliser may further comprise at least one opposing electroacoustic transducer for generating acoustic wave energy in an opposing direction to reduce an extent to which liquid is driven off the at least one piezoelectric substrate prior to nebulisation.

In one or more embodiments, the at least one opposing electroacoustic transducer may be configured to provide an output indicative of a volume of liquid on the at least one piezoelectric substrate.

In one or more embodiments, the output provided by the at least one opposing electroacoustic transducer may be a current.

In one or more embodiments, the at least one opposing electroacoustic transducer may comprise the sensor.

In one or more embodiments, the nebuliser may further include a control means for controlling a size of the nebulised liquid droplets.

In one or more embodiments, the nebuliser may include at least two piezoelectric substrates spaced apart and located in a parallel adjacent relationship.

In one or more embodiments, the droplet size controlling means may be configured to enable pre-setting of a spacing between the at least two piezoelectric substrates to control a thickness of a meniscus of liquid supplied between adjacent substrate surfaces, to thereby control the size of the nebulised droplets.

In one or more embodiments, the droplet size controlling means may be configured to enable pre-setting of a spacing of the at least two piezoelectric substrates from internal walls of the housing to control a thickness of a meniscus of liquid supplied between adjacent substrate surfaces and the inner walls, to thereby control the size of the nebulised droplets.

In one or more embodiments, the droplet size control means may include a liquid film forming structure in fluid communication with the liquid supply conduit and the at least one piezoelectric substrate to control a thickness of a meniscus of liquid supplied to the at least one piezoelectric substrate to thereby control the size of the nebulised droplets.

In one or more embodiments, the liquid film forming structure may include a web, mesh, one or more fibres, or a slot in the liquid supply conduit.

In one or more embodiments there is provided a nebuliser system comprising: a nebuliser as disclosed above, wherein the nebuliser is a first nebuliser; and a second nebuliser.

In one or more embodiments, the first nebuliser comprises a first nebuliser water contacting surface; and the second nebuliser comprises a second nebuliser water contacting surface.

In one or more embodiments, the first nebuliser water contacting surface is transverse to the second nebuliser water contacting surface.

In one or more embodiments, the first nebuliser water contacting surface is the transducer surface or the non transducer surface; and the second nebuliser water contacting surface is a transducer surface of the second nebuliser or a non-transducer surface of the second nebuliser.

According to another aspect of the disclosure, there is provided a method of nebulising a liquid using a nebuliser as described above or the nebuliser system as described above.

The method may include nebulising liquid to form liquid droplets having a size of across a range between 0.1 μm to 100 μm. The smaller droplet sizes between 1 and 5 μm are ideal for applications for the inhalation of therapeutic agents. It is however to be appreciated that liquid droplets of a larger size beyond 10 μm could be formed if required for other applications including fragrances, cosmetics, pesticides, paints or antiseptics.

The method may further include nebulising liquid at a nebulisation rate up to 10.0 ml/min.

The method may further include nebulising the liquid to form liquid droplets having geometric standard deviation (GSD) of <10 μm.

The method may include nebulising liquid including functional or therapeutic agents therein such as pharmaceuticals, plasmid DNA, RNAi, peptides, proteins and cells, or, non-therapeutic agents such as perfume, cosmetics, antiseptics, pesticides or paints. In one or more embodiments, the functional or therapeutic agent may be delivered as a unit dose. In one or more embodiments, the unit dose is determined by the sensor for detecting the volume of liquid on the surface of the substrate.

The use of both the transducer and non-transducer surfaces for fluid delivery and nebulisation in the nebuliser according to the disclosure not only provides a much higher nebulisation rate (1 ml/min and greater, compared to typical 0.1-0.2 ml/min SAW nebulisation rates) but also circumvents undesirable heating due to viscous dissipation of the acoustic wave energy when it is coupled to the materials typically used for fluid delivery in the previous nebulisation configurations (glass, wick, PDMS, etc.), which typically have poor acoustic matching properties. In addition, the configuration of the nebuliser according to the disclosure also may reduce the contact of chemicals and sensitive samples with the electroacoustic transducer. This has advantages of protecting the electrodes of the transducer from harsh chemicals as well as protecting any sensitive biological samples from the intense electric field generated by the electrodes.

### BRIEF DESCRIPTION OF THE DRAWINGS

It will be convenient to further describe the embodiments with reference to the accompanying drawings, which illustrate embodiments of the nebuliser. Other embodiments are possible, and consequently, the particularity of the accompanying drawings is not to be understood as superseding the generality of the preceding description.

In the drawings:

FIG. 1a is a side cross-sectional view of a nebuliser according to one embodiment;

FIG. 1b is a magnified view of the liquid delivery system which, according to one embodiment, constitutes a nib or needle;

FIG. 1c is a side detailed view of a nebuliser according to one embodiment;

FIG. 1d is a detailed side cross-sectional view of another embodiment of a nebuliser;

FIG. 1e is a side cross-sectional view of another embodiment of a nebuliser;

FIG. 2 is a perspective of a platform that holds a piezoelectric substrate for the nebuliser;

FIG. 3a is an orthogonal view of a transducer surface of a nebuliser;

FIG. 3b is an orthogonal view of a transducer surface of another embodiment of the described nebuliser highlighting the perimeter surface of the substrate. As described, compliant absorbent material may be in contact with at least a portion of the perimeter surface of the substrate surfacer highlighted in FIG. 3b;

FIG. 3c is an orthogonal view of a transducer surface of another embodiment of the described nebuliser highlighting coating on the distal end of the transducer surface and areas suitable for patterning;

FIG. 3d is a representative example of the described nebuliser wherein the non-transducer surface of the described nebuliser is partially coated;

FIG. 3e is an orthogonal view of a transducer surface of another embodiment of the described nebuliser highlighting coating on the distal end of the transducer surface of the substrate;

FIGS. 4a and 4b are side cross-sectional views of another embodiment of a nebuliser;

FIG. 5a is a graph of the ejected drop size distribution fora nebuliser without a baffle;

FIG. 5b is a graph of the ejected drop size distribution fora nebuliser with a baffle;

FIG. 6 is a graph showing the mass sensing of Humalog (insulin medication) as a function of frequency;

FIG. 7 is a graph showing atomisation distribution data of a nebuliser according to one embodiment wherein the non-transducer substrate surface is coated with titanium and gold;

FIG. 8 is a representative example of a sensor for detecting volume of liquid on a surface; (a) by standard methods of monitoring RF load; and (b) a sensor;

FIG. 9 is a representative example of a sensor for detecting volume of liquid on a surface in accordance with the described embodiments in operation; wherein the sensor is adapted to detect the presence (ON/OFF) of a liquid and/or the volume of liquid on the surface—where the nebuliser system is adapted for administration of active agents, the volume of the liquid on the surface may be equated to a unit dose for of a given active to be administered;

FIG. 10 is a representative embodiment wherein the nebuliser includes two opposing IDTs, the black square representing the atomisation zone between the IDTs;

FIG. 11 is a representative embodiment wherein the nebuliser includes a structure for containing the fluid on the surface, the dashed-line indicating the position of a gasket, for example, around the atomisation area;

FIGS. 12 a. to d. are representative examples of liquid film forming structures including, for example, a. bundle of fibres or web at the interface and in fluid communication between the substrate and the liquid supply conduit; b. side-view of thin meniscus forming between fibres or web on the substrate, c. a micro slot in the liquid supply conduit, d. a side view of thin film produced by the micro slot in the liquid supply conduit in operation;

FIG. 13 is a representative embodiment of a nebuliser comprising a sealing that provides a liquid-tight seal between a transducer surface and a mount of the nebuliser;

FIG. 14 is a representative embodiment of a nebuliser system comprising a first nebuliser and a second nebuliser that is provided at an angle with respect to the first nebuliser; and

FIG. 15 is a representative embodiment of the nebuliser system of FIG. 14, illustrating a first trajectory of nebulisation and a second trajectory of nebulisation.

### DETAILED DESCRIPTION

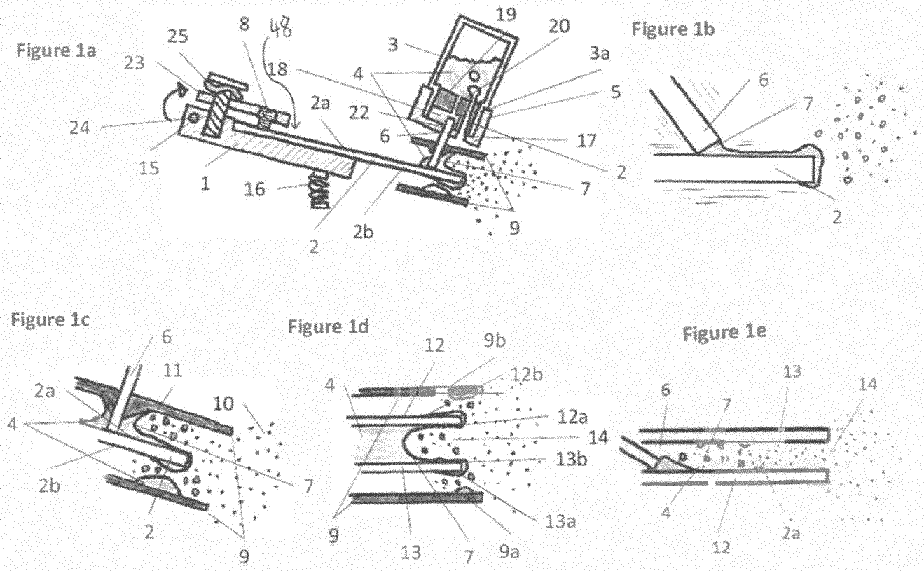

Referring initially to FIGS. 1a and 1c, there is shown a first embodiment of a nebuliser according to the disclosure. The nebuliser includes a mount 1, which supports a piezoelectric substrate 2. The piezoelectric substrate 2 includes a transducer surface 2a upon which is located an electroacoustic transducer 48. The electroacoustic transducer 48 comprises or is in the form of an interdigital transducer (IDT) (not shown). The substrate 2 further includes a non-transducer surface 2b. The non-transducer surface 2b may be disposed or provided on an opposite or reverse surface of the substrate 2 to the transducer surface 2b. As illustrated, the non-transducer surface 2b may be located in a parallel adjacent relationship relative to the transducer surface 2a.

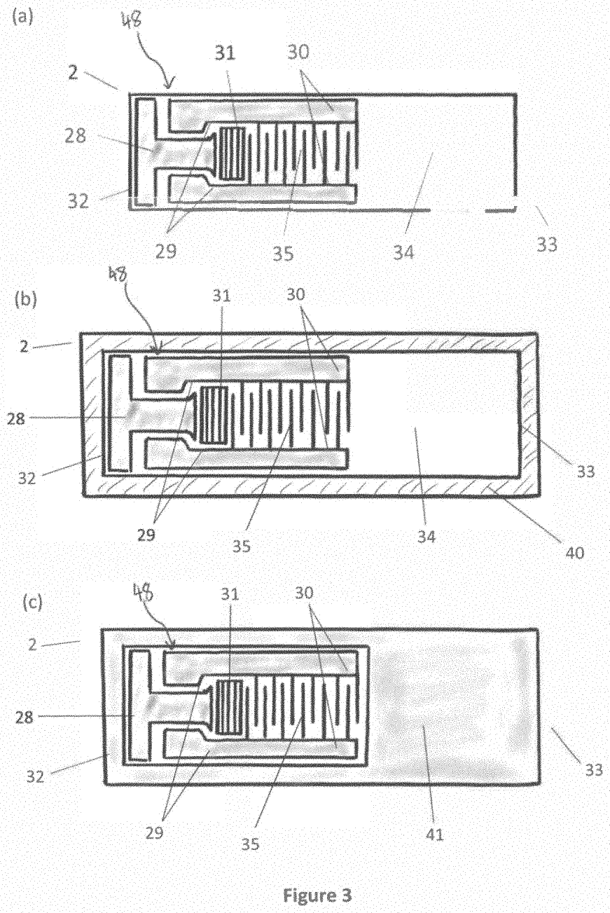

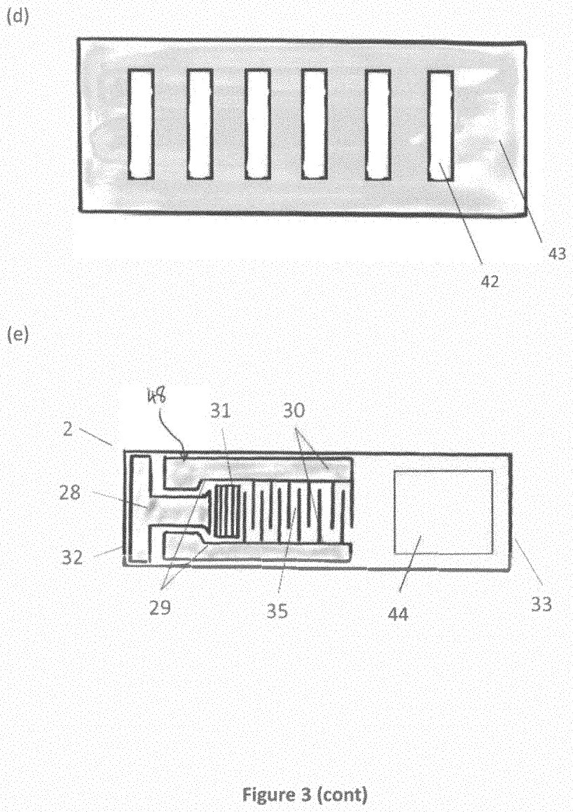

Referring to FIG. 3(a), the electroacoustic transducer 48 comprises or is in the form of one or more interdigital transducers (IDTs) 35. The electroacoustic transducer 48 comprises or spans at least a portion of the substrate 2 and comprises main IDT bars 30. The electroacoustic transducer 48 comprises an electrical contact end 32. The electroacoustic transducer 48 comprises a shield 28. The shield 28 comprises a first elongate portion 60 and a second elongate portion 62. The first elongate portion 60 is substantially perpendicular to the second elongate portion 62. The first elongate portion 60 is substantially perpendicular to the main IDT bars 30. The shield 28 may assist in reducing the extent to which waves (e.g. surface acoustic waves or surface reflected bulk waves, as are described in more detail below) generated by the electroacoustic transducer 48 reach a perimeter 64 of the substrate 2 or the electrical contact end 32. The waves reaching the perimeter 64 of the substrate 2 or the electrical contact end 32 may cause damage and reduce the life of the substrate 2 and/or the electroacoustic transducer 48. The electroacoustic transducer 48 includes bends 29. In particular, the main IDT bars 30 may each comprise one or more bends 29. The bends 29 may assist in reducing the extent to which the generated waves reach a perimeter of the substrate 2 or the electrical contact end 32. The electroacoustic transducer 48 comprises reflector bars 31. The reflector bars 31 may assist in reducing the extent to which the generated waves reach a perimeter 64 of the substrate 2 or the electrical contact end 32.

The nebuliser comprises a liquid supply system. The liquid supply system is configured to supply a liquid to the substrate 2. That is, the liquid supply system is configured to supply liquid to the transducer surface 2a and/or the non-transducer surface 2b. The nebuliser further includes a liquid reservoir 3 within which is accommodated the liquid 4 that is to be nebulised by the nebuliser. In some embodiments, the liquid supply system comprises the liquid reservoir 3. The reservoir 3 can be in the form of a bottle or vial, which may have a threaded neck 3a that can be screwed into a threaded inlet opening 5 provided on a housing (not shown). The liquid supply system may also comprise a supply conduit 6 as is described herein. The supply conduit 6 may be relatively rigid. The nebuliser is shown in its in use position in FIGS. 1a and 1c which thereby allows the liquid 4 to be gravity fed from the reservoir 3 and through the relatively rigid supply conduit 6 in the form of a nib or needle 6. A liquid meniscus 7 is formed at the end of the nib or needle 6 on the transducer surface 2a (FIG. 1b). RF power is supplied to the electroacoustic transducer 48 via electrical contacts 8. This will result in surface acoustic waves (SAWs) being generated in the transducer surface 2a which in turn generates surface reflected bulk waves (SRBWs) that are reflected between the transducer and non-transducer surfaces 2a, 2b. The unique hybrid wave configuration of the SRBWs combined with SAWs allows for liquid 4 to be drawn from the liquid meniscus 7 across the transducer surface 2a. If liquid 4 build-up occurs at the end of the transducer surface 2a, the acoustic wave energy will pull the liquid 4 around the substrate 2 end and onto the non-transducer surface 2b of the substrate 2 where the liquid 4 can also be nebulised. The gravity fed arrangement allows for continuous, self-regulated flow of the liquid to prime the needle or nib 6.

To elaborate further, the supply pump, gravity feed or capillary action in the nib or needle 6 simply acts to prime it. The liquid 4 is then pulled out by the acoustic wave onto the surfaces of the substrate 2, as illustrated in FIG. 1b. In some embodiments, the liquid delivery system, i.e., the nib or needle 6, is in contact with the substrate 2. This is in contrast to the capillary-driven liquid delivery to supply channels etched into the substrate in International Publication No. WO2012/096378 (Panasonic Corp.). Having the acoustic wave drawing out the liquid from the nib or needle 6 onto the substrate 2 avoids flooding since only as much liquid that is nebulised is drawn out onto the device.

The choice of material for the nib or needle 6 may comprise an acoustically reflecting material. Acoustically-absorbing materials tend to absorb and hence dampen the acoustic energy on the substrate 2. Such materials may include metals, polymer or ceramic materials.

Some nebuliser designs use meshes to try to control and maintain uniformity in the size of the nebulised droplets. These nebulisers rely on a piston action generated by ultrasonic or other bulk standing waves to push and pull f through a mesh to generate droplets. Without the mesh, these nebulisers are unable to function since the standing bulk waves will generate an uneven thick film of liquid across the relevant substrate, and subsequently generate uneven and large droplets. Furthermore, such meshes are prone to clogging. The nebuliser embodiments described herein provide surface acoustic waves and surface bulk reflected waves that have a standing and travelling wave component, even on the non-transducer surface 2b of the substrate 2. This pulls the liquid into a thin film across the substrate 2, which results in smaller droplets being uniformly produced.

The housing may include at least one baffle 9, which can, for example, be formed by the wall of the housing. The at least one baffle 9 may be spaced from the transducer surface 2a and may be positioned in a generally parallel and adjacent relationship to the transducer surface 2a. Similarly, the at least one baffle 9 may be spaced from the non-transducer surface 2b and may be positioned in a generally parallel and adjacent relationship to the transducer surface 2b. The at least one baffle 9 may extend along at least a portion of the length of the substrate 2, The baffle 9 provides a simple means of asserting control over the uniformity of the droplet sizes. Larger droplets 11 having a size in the 10 μm to 100 μm order are ejected off the substrate surface 2a with greater momentum than smaller droplets due to the angle (known as the Rayleigh angle) at which the acoustic wave energy couples into the liquid 4. This gives rise to the droplets being ejected as they are nebulised at the same angle. These larger droplets 11 then impact on the surface of the baffle 9 so that they are redirected back to the substrate surface 2a where they are re-fed into the existing liquid feed from the reservoir 3. The liquid that formerly was part of the returned droplets 11 is therefore subjected again to nebulisation. Smaller droplets 10 having a size in the order of around 1 μm, on the other hand, have significantly less momentum and hence do not reach the surface of the baffle 9. Rather, the small droplets 10 are entrained into the airflow out of the nebuliser. A similar droplet size control process also occurs between the non-transducer surface 2b and the corresponding baffle surface 9 adjacent to the non-transducer surface 2b.

FIG. 1d shows another embodiment of the nebuliser according to the disclosure utilising at least two piezoelectric substrates 12, 13 supported in a stacked configuration within the nebuliser. More than two piezoelectric substrates can also be stacked in parallel and adjacent locations within the nebuliser. Each piezoelectric substrate 12, 13 will have a similar arrangement to the embodiment shown in FIGS. 1a and 1c with an electroacoustic transducer 48 located on a transducer surface 12a, 13a of each substrate 12, 13 to allow for acoustic wave energy to be generated within each substrate to thereby draw nebulised liquid supplied to both the substrate surface 12a, 13a and parallel adjacent (or reverse) non-substrate surface 12b, 13b of each substrate 12, 13. The housing also includes a lower baffle 9a that is located parallel and adjacent to the transducer surface 13a of the lower substrate 13, which assists in droplet size control as previously described. A similar effect occurs between the non-transducer surface 12b of the upper substrate 12 and the baffle 9b opposite to that surface. The orientation of the transducer 12a, 13a and non-transducer 12b, 13b surfaces of the two substrates 12, 13 may be interchanged as long as the respective transducer surfaces 12a, 13a and non-transducer surfaces 12b, 13b are reverse, or parallel and adjacent, to one another. This arrangement, however, provides a further means for controlling the uniformity of the droplet size. Liquid is also trapped between the interstitial space 14 between the two substrates 12, 13, and between the transducer surface 13a of the lower substrate 13 and the lower baffle surface 9a, and between the non-transducer surface 12b of the upper substrate 12 and the upper baffle surface 9b. The thickness of the liquid meniscus 7 is a parameter used in controlling the droplet size. Therefore, adjustment of the relative spacing between each substrate 12, 13 and baffle surfaces 9a, 9b allows the meniscus thickness to be controlled, thereby providing uniformity in the nebulised droplet size. This configuration therefore allows for the droplet size to be controlled by adjusting the above noted spacing. It is also envisaged that multiple droplet sizes could be obtained by having multiple spacings.

FIG. 1e shows another embodiment of the nebuliser according to the disclosure utilising at least two piezoelectric substrates 12, 13 supported in a stacked configuration within the nebuliser. Like the embodiment described in FIG. 1d, liquid is trapped between the interstitial space 14 between the two substrates 12, 13. Unlike in FIG. 1d, the liquid meniscus 7 need not be in contact with both substrates 12 and 13. Furthermore, the nib or needle 6 may, in an embodiment, be in direct contact with the surface of one of the substrates 12 to deliver the liquid 6. In another embodiment, the nib or needle 6 may not be in contact with the surface of the substrate 12, but may be positioned such that the liquid 6 is delivered to be in contact with the surface of the substrate 12. It is envisaged that the at least two piezoelectric substrates 12, 13 may be the same or may be different. For example, one or more of the substrates may be patterned as described in detail below to provide further control of the nebuliser output parameters.

Furthermore, in view of the arrangements in FIGS. 1d and 1e, for example, a higher nebulisation rate can be provided because there are now multiple substrate surfaces from which nebulisation can occur. An adjacent substrate surface can also act as an active baffle where spurious large droplets ejected from one substrate surface are collected onto the surface of an adjacent substrate and re-nebulised until smaller droplets are produced. This approach may be considered active substrate baffling rather than a passive physical baffle provided by a housing inner wall. This system may be enhanced by promoting standing waves or regions of standing waves using the aforementioned techniques.

The same piezoelectric substrate 2, 12, 13 and electroacoustic transducer 48 can also be triggered at a lower frequency corresponding to the fundamental thickness mode (BAW) of the substrate (around 3.5 MHz for a 500 μm thick substrate) to employ a sensing functionality. The rationale for using the thickness mode for sensing is because single crystals such as, but not limited to the 128 YX lithium niobate piezoelectric crystal used, naturally have a high-quality factor Q on the order of between 10⁴ to 10⁶. Therefore, such a platform can simultaneously perform both efficient nebulisation as well as efficient mass sensing with a limit of detection down to 10 ng. Both functions can be achieved with the same electrode patterns unlike other known devices that incorporate different electrode patterns and/or require completely different additional electrodes for different microfluidic functions. These other devices are triggered at a particular resonant frequency, whereas the nebuliser embodiments described herein provide an electrical circuit that both enables nebulisation and sensing functionality. That is, using the same circuit, two modes are enabled, a first mode for nebulisation and a second mode for sensing. In some embodiments, the sensing mode may be as described in International Publication No. WO2015054742A1, the content of which is incorporated herein by reference.

Therefore, the nebuliser according to the described embodiments can add the functionality of sensing mass residual during nebulisation in order to determine, by subtraction from the total dose delivered, the actual dose that is administered to the user. Furthermore, the nebuliser embodiments described herein advantageously do not require multiple storage parts (e.g. fluid storage parts) to enable the sensing functionality, which is advantageous over other devices which require multiple storage parts.

In the above embodiment of FIGS. 1a and 1c, the liquid 4 is gravity fed to a nib or needle 6. The nib or needle 6 presses onto the end of the transducer surface 2a, bringing liquid 4 into contact with the transducer surface 2a where it can be atomised into droplets 10, 11. Robust contact between the nib or needle 6 is achieved by displacing the mount 1 towards the nib or needle 6 which is pre-loaded with a force and exerts a constant pressure under displacement (not shown). In one embodiment, the pre-loaded force is achieved by fixing the mount 1 to a cantilever, or by configuring the mount 1 with a pivot 15 and a resilient member in the form of a spring 16 arrangement that are fixed to the housing (not shown), for example. The displacement of the mount 1 caused by the pressing of the nib or needle 6 onto the substrate 2 allows constant pressure and contact between the end of the nib or needle 6 and the transducer surface 2a to be realised, and for a meniscus 7 to form and be sustained. This meniscus 7 provides pressure equal to that of the sealed reservoir 3 so that liquid does not flow freely from the reservoir 3 onto the substrate. The capacity for the mount 1 to be displaced and exert pressure means that a rigid nib or needle 6 can be used in direct contact with the substrate effectively. Referring to FIG. 1b, the nib or needle resonates with the acoustic wave energy, allowing the acoustic wave energy to draw liquid 4 from the nib or needle 6 across the substrate surface 2. During nebulisation of the liquid 4, the loss of liquid 4 will diminish the meniscus 7. The negative pressure that arises then draws further liquid 4 through the nib or needle 6 to replenish the meniscus 7. When the relative pressure of the reservoir 3 is sufficiently low due to the outflow of liquid 4 through the nib or needle 6, an air bubble will enter the reservoir 3 via the inlet hole 17 to balance the pressure and allow liquid 4 to be drawn by the nib or needle 6. This process will continue until the reservoir 3 is exhausted. It is envisaged that multiple nibs or needles could be used to increase flow rate and increase the reliability of the system. It is, however, also envisaged that a pressure release valve be used to provide a controlled flow of liquid onto the transducer surface 2a. It is further envisaged that the end of the substrate 2 be submerged in a meniscus, where the liquid is provided by a closely situated orifice. Alternatively, it is envisaged that an active pumping system, such as a syringe or peristaltic pump be used to actively feed liquid onto the substrate surface 2a. An active pumping system may be preferred in situations where liquid having a high surface tension and/or high viscosity needs to be delivered to the transducer surface 2a.

A flow regulator 19 may also be used in conjunction with the above described gravity feed system, adjacent orifice, or active pumping system. It is also envisaged that a flow regulator 19 works in a similar fashion to a fountain pen. Such an arrangement is shown in FIG. 1a where fluid within a reservoir 3 flows into an inner chamber 18 via a flow regulator 19. The flow regulator 19 includes a liquid outlet passage 20 through which liquid 4 can pass, and an air inlet passage 21 connected to the reservoir 3. The flow regulator 19 therefore provides a steady feed of liquid 4 that would otherwise be disrupted by the release of air bubbles that enter through the inlet passage 21 to thereby balance the air pressure externally and within that reservoir 3. The liquid 4 is delivered to an inner chamber 18. The inner chamber 18 connects to the nib or needle 6, and has a peripheral opening 22 within which is accommodated the nib or needle 6. The nib or needle 6 is therefore constantly wetted by the liquid 4.

The electrical contact end of the substrate 2 is pressed and in direct contact with the mount 1 in order to dissipate localised heating that can damage the substrate 2. This pressing can be achieved by applying pressure through contact cantilevers 23 with broad electrical contacts 8 embedded in them, for example—broad electrical contacts 8 also mitigate damaging arcing between the electrical contacts 8 and the substrate 2 under the high voltages that occur during nebulisation. Pressure to the contact cantilever 23 bases can be applied via magnetic attraction effects, or by using a screw 24 to push down spring washers 25, for example. Alternatively, pressure may be applied through spring loaded electrical contacts. Furthermore, it is envisaged that a conductive material may be directly bonding to the electroacoustic transducer 48 as an alternative to electrical contacts. A heat sinking surface (not shown), which could be integrated into the mount 1, can also be utilised by the pressing of the nib or needle 6 onto the parallel substrate 2, which can then remain in contact with the heat sink and cool the substrate 2 during nebulisation. This heat sink may also feature geometry that retains a small amount of excess liquid in contact with the nebulisation end of the substrate 2 to further increase the robustness of the system while nebulisation is occurring. The mount 1 may also be made of a conductive material such as metal, which will allow the ready discharge of excess pyroelectrically induced charge. This reduces the chance of damaging arcing across the substrate 2, increasing the life of the substrate 2.

It is recognised that one of the challenges for SAW nebuliser systems in administration of active agents, including inhaled medication, is accurate, measurable dosage delivery to ensure the correct dose is received by the patient for therapeutic effect. As such, in still other embodiments, the nebuliser according the described embodiments may include one or more sensors to detect the volume of liquid on the surface of the substrate by measuring a change in current across the nebuliser system. In one or more embodiments, the nebuliser system includes an electronic circuit and at least one piezoelectric substrate. In some embodiments, the sensor, to detect the volume of liquid on the surface of the substrate, measures a change in direct current (DC) across the nebuliser system. It is understood that the electronic circuit may include at least one printed circuit board (PCB). Therefore, the same circuit (i.e. the circuit of the nebuliser comprising the electroacoustic transducer) may be used for both nebulisation and sensing.

In some embodiments, the electroacoustic transducer 48 is configured to indicate a volume of liquid on the at least one piezoelectric substrate 2. That is, the electroacoustic transducer 48 is configured to provide an output indicative of the volume of liquid on the at least one piezoelectric substrate 2, as is described herein. The output may be a current passing through the electroacoustic transducer 48. The electroacoustic transducer 48 is therefore capable of both nebulising the liquid and sensing a volume of liquid on the at least one piezoelectric substrate 2.

The sensor disclosed herein can function independently of the size or shape of the electrical circuit of the nebuliser (e.g. the electroacoustic transducer 48, 50). The sensor can also function regardless of the size or shape of the substrate 2. These are both significant advantages provided over other nebulisers.

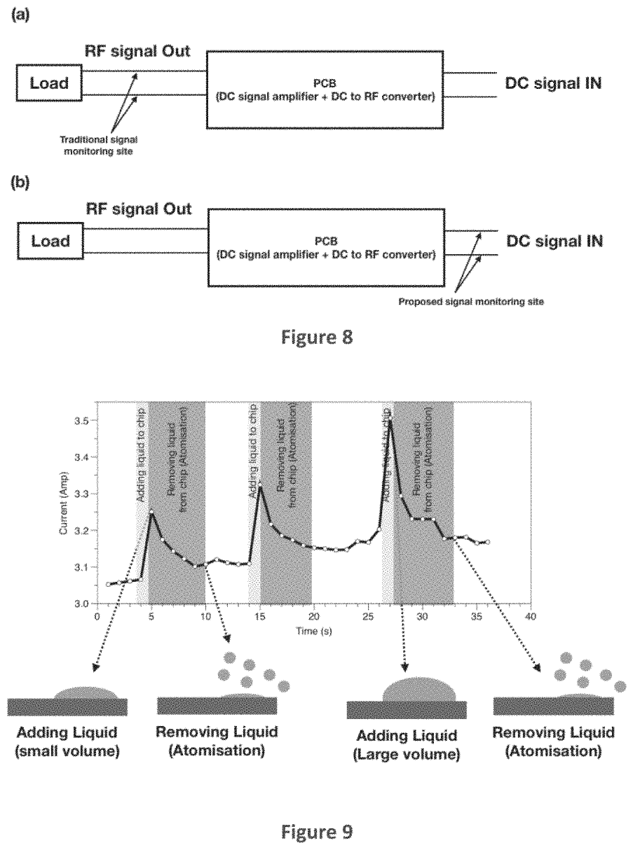

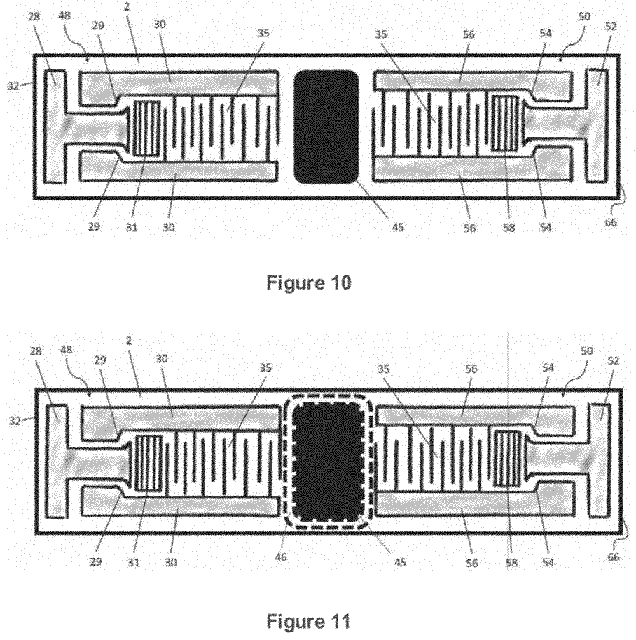

The approach described herein measures the DC current input across the entire circuitry (shown in FIG. 8b). Considering the many variables downstream from the DC signal into the PCB all the way to the load this is not straightforward. It is envisaged that the PCB design can be constructed carefully to eliminate any fluctuations for the current input, except for the change in the load, past the RF signal output. Shown in FIG. 9 is an example where the overall current fed into the entire PCB circuitry is shown to increase as soon as liquid is brought into contact with the chip and then reduced when the fluid is removed (atomised). This represents a whole systems approach to fluid detection, where the PCB and substrate/load are considered as one entity. This approach thus provides a remarkably simple approach for fluid monitoring through DC current fluctuation and contrasts existing systems which instead measure downstream RF (shown in FIG. 8a). It is envisaged that DC current fluctuation can, for example, be easily monitored within an onboard current counter (i.e. a small component added to the PCB). It is appreciated that such a sensor, for example, adapted to measure DC across the entire circuitry, can not only act as a means for measuring the fluid volume on the surface of the substrate but can also optionally act as a switch for the atomisation process (i.e. ON/OFF) once the liquid amount is below or above a desired threshold. Accordingly, in one or more embodiments, the nebuliser of the described embodiments may further comprise a control switch responsive to the sensor for controlling the operation of the nebuliser. In one or more other embodiments, the nebuliser may further comprise a control valve responsive to the sensor for controlling the fluid flow to the substrate.

The sensor is configured to detect the current fed into the PCB circuitry (e.g. to the electroacoustic transducer to nebulise the liquid) to provide an indication of an amount of liquid 4 on the substrate 2. The nebuliser, or an associated component (e.g. a computing device) can use a reading output by the sensor that is indicative of the amount of liquid on the substrate 2 to determine an amount of liquid that has been nebulised over a certain time period. The amount of liquid that has been nebulised can be equal to, or associated with, an amount of liquid delivered from the nebuliser (e.g. to a user of the nebuliser). This can, for example, be a volume or a mass of liquid that has been nebulised. This is advantageous over other nebulisers, as other nebulisers may only be capable of determining the presence or lack of liquid, rather than an amount that has been nebulised.

In still other embodiments, it is envisaged that this approach can be expanded to other types of fluids with different conductivity or viscosity, and be used to monitor properties of the fluids.

It is further appreciated that the sensor to detect the volume of liquid on the surface of the substrate may be adapted for accurate, measurable dosage delivery of active agents, including inhaled medication. In one or more embodiments, the administration of a single unit dose may be determined by a sensor for detecting the volume of liquid on the surface of the substrate. In still other embodiments, there is provided a method for administration of a functional or therapeutic agent as a single unit dose.

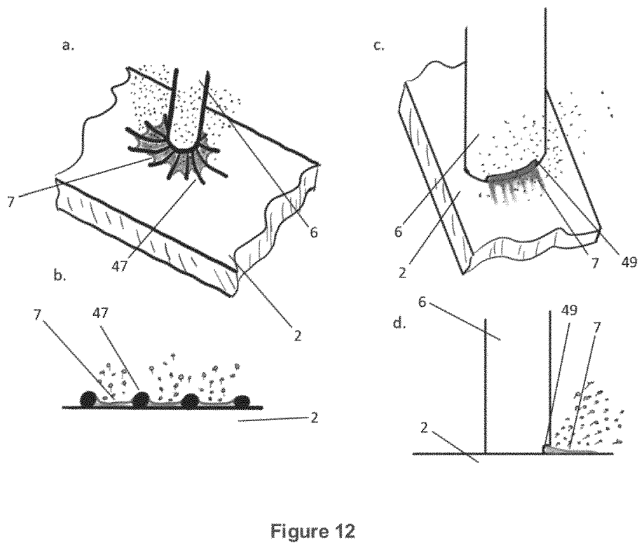

It is appreciated that loss of atomising liquid from the surface, side or end of the substrate remains a challenge for SAW nebuliser systems in administration of active agents, including inhaled medication. This may occur, for example, where the acoustic waves drive the liquid off the surface prior to atomisation. Loss of atomising liquid from the chip surface may alter dosage rates or render inhalation therapy less effective, which can adversely affect the subject. As such, in one or more embodiments there is provided a nebuliser further comprising at least one opposing electroacoustic transducer 50 for generating acoustic wave energy in an opposing direction to prevent liquid from being driven off the surface of the substrate prior to nebulisation. The electroacoustic transducer 48 may be referred to as a first electroacoustic transducer. The at least one opposing electroacoustic transducer 50 may be referred to as a second electroacoustic transducer. The at least one opposing electroacoustic transducer 50 comprises or is in the form of one or more interdigital transducers (IDTs) 35. The IDTs 35 of the at least one opposing electroacoustic transducer 50 may be similar or the same as the IDTs 35 described with reference to the electrostatic transducer 48. The at least one opposing electroacoustic transducer 50 comprises or spans at least a portion of the substrate 2 and comprises main IDT bars 56. The electroacoustic transducer 48 comprises an electrical contact end 66. The electrical contact end 66 may be similar to or the same as the electrical contact end 32 previously described. The main IDT bars 56 may be similar to or the same as main IDT bars 30 described with reference to the electrostatic transducer 48. The at least one opposing electroacoustic transducer 50 comprises a shield 52. The shield 52 may assist in reducing the extent to which waves (e.g. surface acoustic waves or surface reflected bulk waves) generated by the at least one opposing electroacoustic transducer 50 reach a perimeter of the substrate 2 or the electrical contact end 66. The waves reaching the perimeter of the substrate 2 or the electrical contact end 66 may cause damage and reduce the life of the substrate 2 and/or the electroacoustic transducer 50. The shield 52 may be similar or the same as the shield 28 described with reference to the electrostatic transducer 48. The at least one opposing electroacoustic transducer 50 comprises bends 54. In particular, the main IDT bars 30 may each comprise one or more bends 54. The bends 54 may assist in reducing the extent to which the generated waves reach a perimeter of the substrate 2 or the electrical contact end 66. The at least one opposing electroacoustic transducer 50 comprises reflector bars 58. The reflector bars 58 may assist in reducing the extent to which the generated waves reach a perimeter of the substrate 2 or the electrical contact end 66. The reflector bars 58 may be similar to or the same as the reflector bars 31 described herein. FIG. 10 provides a representation of such an arrangement of opposing electroacoustic transducers 48, 50 (which may be in the form of IDTs), where features such as shields 28, 52, bends 29, 54, main IDT bars 30, 56 and reflector bars 31, 58 are added accordingly.

It has surprisingly been found that atomising fluid between opposing electroacoustic transducers 48, 50 may advantageously prevent excess fluid from being driven off the surface, a distal end or a side of the substrate 2 by acoustics. Advantageously, it is envisaged that such a configuration of opposing electroacoustic transducers 48, 50 may provide a stable atomisation zone 45 between the opposing electroacoustic transducers 48, 50, effectively increasing an atomisation area and, in turn, the potential atomisation rate. Providing opposing electroacoustic transducers 48, 50 on either side of the stable atomisation zone 45, the nebuliser may generate opposing acoustic waves which interact. That is, the at least one opposing electroacoustic transducer 50 may generate acoustic waves that are equal in magnitude to the acoustic waves generated by the electroacoustic transducer 48, but that are generated in an opposing direction. The opposing acoustic waves may provide the stability in the stable atomisation zone 45. In some cases, if the at least one opposing electroacoustic transducer 50 were not provided, liquid in the stable atomisation zone 45 may be driven away from the electroacoustic transducer 48 by the acoustic waves generated by the electroacoustic transducer 48. The liquid may therefore fall off the substrate 2 rather than nebulising. The above-described embodiment can be configured to ensure the liquid is maintained within the stable atomisation zone 45 until nebulisation, therefore providing a solution to this problem.

Degradation of the substrate 2 can be reduced by providing (e.g. bonding) a material that is relatively acoustically non-absorbent to the substrate 2. In some embodiments, the acoustically non-absorbent material is selectively provided to the substrate. For example, the acoustically non-absorbent material can be bonded to the substrate over the stable atomisation zone 45. This may reduce degradation of the stable atomisation zone 45. The acoustically non-absorbent material may be a metal. The metal can be electroplated over the substrate 2, for example, to the stable atomisation zone 45.

In still other embodiments, such as that shown in FIG. 11, there is provided a nebuliser wherein the substrate 2 further comprises a containing barrier structure 46. The containing barrier structure 46 is for containing liquid and/or preventing or reducing loss of liquid applied to the surface (i.e. the transducer surface 2a) prior to nebulisation. By way of example, such a containing barrier structure 46 may include a lip, a wall, a gasket, a deposited raised film or combinations thereof. FIG. 11 provides a representation of such a containing barrier structure 46 for containing liquid and/or preventing or reducing loss of liquid applied to the surface (i.e. refer to dotted region, which may include a gasket on the surface of the substrate). Advantageously, the containing barrier structure 46, which may extend around the stable atomisation zone 45, may also allow for the isolation of the stable atomisation zone 45 from the rest of the system, including the electroacoustic transducers 48, 50. This has the added advantage of protecting the other elements of the system from potentially damaging fluid contact and fouling. In one embodiment, a solid hydrophobic gasket is pressed in contact around the edges of the stable atomisation zone 45. It is envisaged that such a containing barrier structure 46, such as a gasket, would prevent or reduce the likelihood of fluid leaving the stable atomisation zone 45 and would not significantly dampen the acoustic radiation.

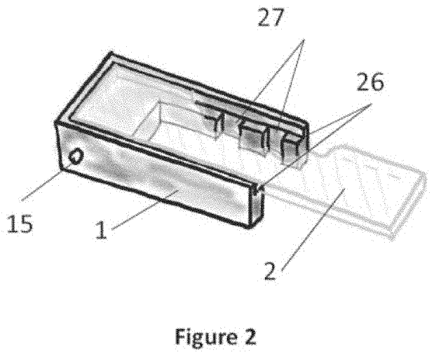

Referring now to FIG. 2, the mount 1 holds the substrate 2 along its side edges on a narrow shelf 26 so that if any wetting occurs between the mount 1 and the substrate 2 the acoustic wave energy will not be damped as it travels along the substrate 2. There are also provided gaps 27 along the narrow shelf 26 of the mount 1, which prevent liquid 4 from creeping up the substrate 2 between the contact of the substrate 2 and the mount 1.

Referring again to FIG. 3(a), the transducer surface 2a possesses surface features, such as the shield 28, bends 29 in the main IDT bars 30, and reflector bars 31 at the electrical contact end 32, that disrupt the progression of acoustic wave energy and encourage reflection and absorption of potentially damaging acoustic wave energy at the electrical contact end 32. Reflected acoustic wave energy aids in the nebulisation of liquid at the nebulisation end 33 of the substrate 2. Bare surface 34 lies between the end of the main IDT bars 30 and the nebulisation end 33 of the device to mitigate contact between the nebulising liquid and the IDTs 35.

In another embodiment, the described nebuliser may further comprise a compliant absorbent material in contact with at least a portion of the perimeter surface of the substrate. For example, the perimeter surface of the substrate is highlighted as a hashed region 40 in FIG. 3(b). It is appreciated that the compliant absorbent material may be in contact with at least a portion of the perimeter surface 40 highlighted in FIG. 3(b). It has surprisingly been found that the durability of the chip may be enhanced by the addition of a compliant material in contact with at least a portion of the perimeter surface of the substrate. Without wishing to be bound by theory, it is considered that the addition of a compliant material may disperse or reduce excess vibrations in and/or on the chip. Furthermore, it is considered that the addition of a compliant material may prevent overheating or localized superheating in and/or on the substrate. This reduces the rate of substrate failure, providing increased reliability and use from the nebuliser without damage or failure. For example, suitable compliant materials, may include pastes, tapes, or compliant solids. In an embodiment, the compliant material is adhesive tape. In an embodiment, the compliant material is silicone rubber. In an embodiment, the compliant material is thermal paste. In an embodiment, the compliant material comprises a portion of the housing in contact with the perimeter of the chip.

In an embodiment, the compliant absorbent material may be in contact with at least a portion of the perimeter of the distal end of the substrate. In an embodiment, the compliant absorbent material may be in contact with at least a portion of one or more sides of the surface of the perimeter of the substrate. In an embodiment, the compliant absorbent material may be in contact with a portion of one or more sides and a portion of the distal end of the substrate. In particular, placement around at least a portion of the perimeter surface allows acoustic radiation in the atomisation region of the substrate to be sufficient to achieve atomisation.

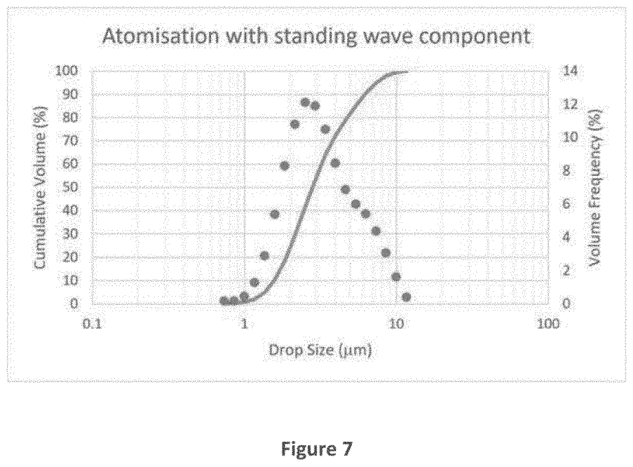

It has further been found that coating at least a portion of the non-transducer side of the substrate may alter wave reflections and the standing wave ratio (SWR). In one embodiment, the coating may comprise one or more metals. In an embodiment, the coating is formed from titanium, gold, aluminium, chromium and combinations thereof. The inventors have surprisingly found coating at least a portion of the non-transducer surface of the substrate with one or more metals may reduce overheating. Additionally, the inventors have surprisingly found that coating at least a portion of the non-transducer surface of the substrate provides a degree of control and/or the ability to tune the standing wave and traveling wave components in SAW, SRBW and combinations thereof. It has surprisingly been found that solid coatings or partial coatings effect the travelling and standing wave components present on and in the substrate. A representative example is shown in FIG. 3(d), that is, the non-transducer surface 43 of the substrate being partially coated 42. The standing wave ratio may be further modified by adjusting parameters such as coating hardness, thickness, and/or roughness. It has been observed that adjusting the standing wave ratio between 1 and infinity can increase the stability and atomisation rates of the substrate. By way of example, atomisation distribution data is represented in FIG. 7, wherein non-transducer substrate surface was coated with titanium and gold. As a result of the coating, the overall droplet distribution was tighter as measured by geometric standard deviation (GSD). By comparison, when an uncoated chip is used, two separate peaks in droplet distribution of nebulised fluids are typically observed. It is considered that this results from promotion or preference of travelling wave components rather than standing wave components in this system. Conversely, where the chip is coated, promotion or preference for standing wave components rather than traveling wave components is observed. By modifying the ratio of travelling and standing wave components, parameters including the droplet size and geometric standard deviation may be controlled or adjusted. These parameters are further described below. In one or more embodiments, the described nebuliser may utilise traveling wave components, standing wave components and/or combinations thereof. In one or more further embodiments, the described nebuliser may utilise standing wave components in SAW, standing wave components in SRBW, traveling wave components in SAW, traveling waves components in SRBW, permutations and combinations thereof.

In addition to coatings applied to the non-transducer surface, the inventors have surprisingly found coating at least a portion of the transducer surface of the substrate with one or more metals may reduce overheating. In particular, the inventors have found that where at least a portion of the transducer surface further includes a coating at the distal end of the substrate, chip failure due to overheating or pyroelectric failure is reduced or eliminated providing a more efficient and robust system. In one embodiment, the coating on the transducer surface may comprise one or more metals. In an embodiment, the coating is formed from biocompatible metals, including titanium, gold, and combinations thereof. Representative examples are shown in FIGS. 3(c) and 3(e), that is, wherein the entirety of the transducer surface of the substrate includes a coating 41 (FIG. 3c) and wherein at least a portion of the transducer surface of the substrate includes a coating 44 at the distal end of the substrate (FIG. 3e).

In another embodiment, the described nebuliser may further comprise patterning of conductive material on a portion of the substrate surface. As used herein, the terms “patterning” and “patterned” and variations thereof, refers to techniques such as photolithography, which transfer a geometric pattern on to a given substrate. Such techniques are typically used for patterning in the chip industry. Generally, a coating, especially a metal coating as described, is applied and the surface subsequently patterned by lithography or other means. In an embodiment, the transducer substrate surface is patterned. In another embodiment, the non-transducer substrate surface is patterned. It has surprisingly been found that the addition of patterning (in areas other than functional areas of the transducer surface of the substrate) may aid in dissipating or reducing localised superheating and/or pyroelectrically induced charge. It is further understood that the non-transducer surface of the substrate may alternatively or additionally may be patterned. FIG. 3(c) highlights the functional areas of the transducer surface of the substrate (including the main IDT bars 30, the IDTs 35, shield 28, bends 29, reflector bars 31). One of the areas of the transducer surface of the substrate suitable for patterning includes the coated surface 41 highlighted in FIG. 3(c) in grey. A skilled person would understand that such patterning may be placed in any region of the surface of the chip which still enables the device to function as a nebuliser.

In addition, it has been found that adjustments in the standing wave ratio may also be achieved by positioning multiple sets of IDTs such that the resultant waves interact. By way of example, it is envisaged that patterning of IDTs may disrupt destructive acoustic waves and reduce unwanted overheating for example, which in turn increases the reliability of the resultant chip. Furthermore, in an embodiment, the substrate may be patterned or coated in such a way to provide discrete regions wherein either standing or traveling waves are promoted. It is envisaged that such an arrangement provides further tunability in a range of output parameters of the nebulised liquids.

While embodiments utilising a needle or a nib have been described, still other embodiments are envisaged wherein the at least one supply conduit may include a wick or a microchannel. The choice of a specific supply conduit may be dependent, in part, on how the conduit operates in combination with other features of the nebuliser system.

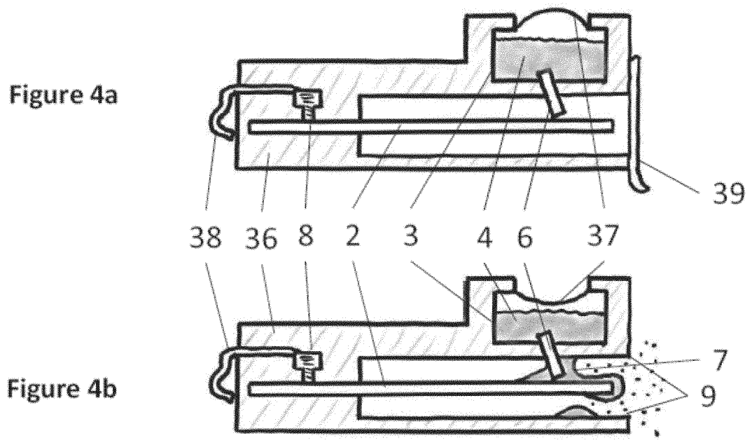

FIGS. 4a and 4b depict another embodiment of the nebuliser. This arrangement integrates the substrate 2 and other key components into a single integrated housing or cartridge 36, which can be interfaced with an external housing that features the appropriate electrical system and flow chamber of a nebuliser (not shown), and used as a single or multiple dose cartridge 36 that can be disposed of after use. The reservoir 3 can be formed from a cavity in the cartridge 36, where one surface of it can be a deformable blister or button 37 that can be depressed; this can displace liquid inside the reservoir and serve to prime the liquid 4 in the needle or nib 6, or deposit a full dose of liquid 4 onto the substrate 2 to form a meniscus 7—other means of displacing the liquid 4 such as a syringe plunger are also possible. FIG. 4a represents the system before the blister 37 is depressed and the liquid 4 deposited, and FIG. 4b shows the system after the blister 37 has been depressed, causing liquid 4 deposition. RF power can be supplied to the substrate via exposed spring contacts 38 that are connected to the broad electrical contacts 8 that are in contact with the substrate 2. The exposed spring contacts 38 allow the cartridge 36 to be interfaced with an external body that can house the appropriate nebuliser electrical systems and flow chamber (not shown). The surrounding surfaces, such as the surrounding parallel surfaces, around the substrate 2 act as baffle surfaces 9 to control drop size and recirculate excess liquid 4. The cartridge can be protected by a seal 39 that can be breached or removed before liquid 4 is nebulised or when the cartridge 36 is interfaced with the external body of the nebuliser. This cartridge can incorporate any combination of the features described and shown in FIGS. 1a, 1c, 1d, 1e, 2 and 3a, 3b, 3c or 3d.

The presented circuit is a miniaturised handheld circuit running at high frequency (10 MHz). The main reason for overcoming the miniaturisation bottleneck, where alternative Radio Frequency (RF) circuits are bulky, is due to the simplicity of the circuit. Unlike common RF circuits where most critical components commonly and intuitively rely on digital data and programming to track the target frequency and trigger various add-on components such as sensor driver, powering buttons, etc., this circuit utilises a robust, stable, fixed, single frequency regardless the loading nature on the circuit. In addition, the circuit is capable of sensing user breathing patterns to drive the nebuliser and/or run by a triggering button, it maintains only an analogue data transfer and actuation for the entire circuit.

The circuit, although small and compact, provides dual triggering methods by either, 1—continuously pressing or toggling a button or 2—‘smart’ triggering via user inhalation, where the triggering time is predetermined, thus accommodating a user inhaling for too long. Therefore, this allows for a precise administration time and therefore known dosage.

The above-mentioned counter-intuitive circuit design approach, utilising analogue data transfer working in RF domain, has allowed the circuit to be driven via a small 11.1V (3 cell) Lithium-polymer battery.

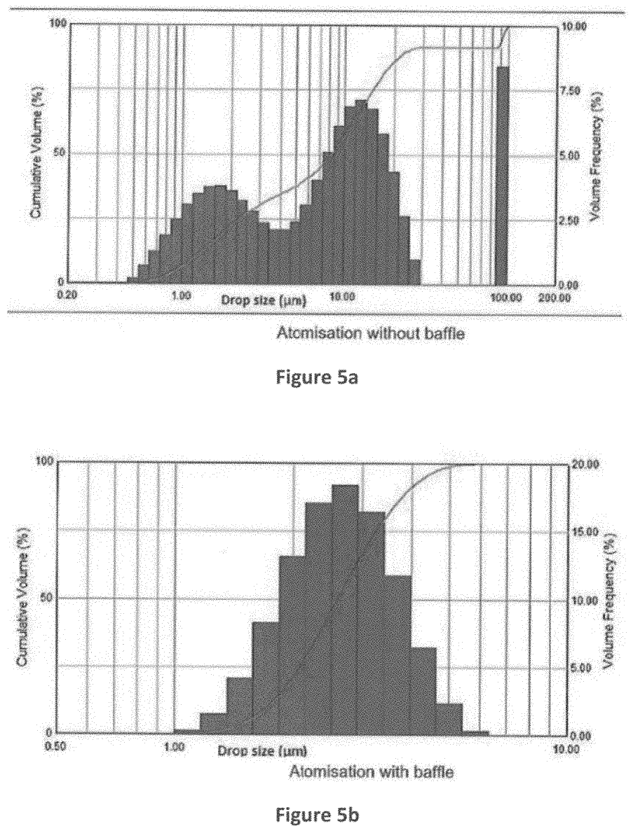

FIG. 5a shows the ejected drop size distribution without the use of a baffle 9. The graph shows that a large proportion of the droplets have a size in the 10 μm to 100 μm range. FIG. 5b shows the ejected drop size distribution when a baffle 9 is used. That graph shows that large droplets in the 10 μm to 100 μm size are minimised.

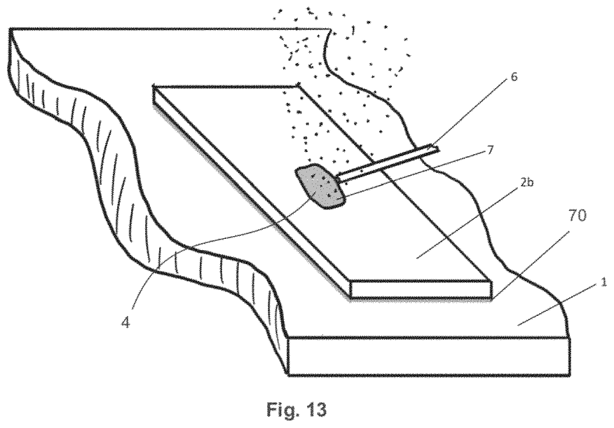

In still other embodiments, the droplet size control means may further include a liquid film forming structure 47. The liquid film forming structure 47 may be in fluid communication with the liquid supply conduit 6 and the substrate 2 to control the thickness of the meniscus 7 of the liquid supplied to the substrate surface to thereby control the size of the nebulised droplets. In still other embodiments, the liquid film forming structure 47 is at the interface between the liquid supply conduit 6 and the substrate 2 to control the thickness of the meniscus 7 of the liquid supplied to the substrate surface to thereby control the size of the nebulised droplets. In a further embodiment, the liquid film forming structure 47 is an integral part of the substrate 2 or is directly bonded to the substrate 2. This may be achieved, for example, through electroplating. The liquid film forming structure 47 may therefore be an electroplated structure. FIGS. 12a to 12d show a number of embodiments of the liquid film forming structure 47. The liquid film forming structure 47 may include a web, mesh, one or more fibres, a slot in the liquid supply conduit or combinations. Structures can be in contact with the device surface that encourage the formation of fluid films that allow for drop-size control. FIGS. 12a and 12b show one embodiment where a bundle of hard flexible fibres 51 is pressed and splayed across the surface of the substrate 2 and acts as a fluid conduit 6. The fibres 51 encourage the formation of thin fluid films that promote the formation of small droplets that are ideally sized for deep lung penetration. As shown in FIGS. 12c and 12d, fluid conducting structures with small openings at their ends, like micron-sized high-aspect ratio slots 53, can be brought into contact with the device surface and deliver fluid via the small openings and encourage the formation of thin films and small droplets in turn.

In some embodiments (not shown), the non-transducer surface 2a, 2b, 12b, 13b comprises one or more electroacoustic transducers. Such a non-transducer surface may be referred to as a second transducer surface. These embodiments may comprise one or more electroacoustic transducers similar to or the same as the electroacoustic transducer 48 described previously. These embodiments may comprise at least one opposing electroacoustic transducer like that described previously. The non-transducer surface (or second transducer surface) of these embodiments may therefore also comprise a stable atomisation zone 45 as previously described. The non-transducer surface (or second transducer surface) of these embodiments may also comprise a containing barrier structure 46 as previously described.

FIG. 13 illustrates another embodiment of the nebuliser. As previously described, the unique hybrid wave configuration provided by the nebuliser in the form of SRBWs combined with SAWs allows for liquid 4 to be nebulised from both the transducer surface 2a and non-transducer surface 2b. In some embodiments, the nebuliser is configured such that the liquid is applied to the non-transducer surface 2b. The liquid 4 is then nebulised from the non-transducer surface 2b. In the embodiment of FIG. 13, the liquid supply conduit 6 is configured such that liquid 4 is provided by the liquid supply conduit 6 to the non-transducer surface 2b. The liquid 4 forms a meniscus on the non-transducer surface 2b and the liquid 4 is atomised from the non-transducer surface 2b by activation of the electroacoustic transducer 48 (not shown in FIG. 13) and the at least one opposing electroacoustic transducer 50 (if provided).

The transducer surface 2a is bonded to the mount 1. In some embodiments, one or more edges of the substrate 2 are bonded to the mount 1 with a sealing 70. The sealing 70 bonds to the substrate 2 and to the mount 1 to seal the one or more edges of the substrate 2 to the mount. In some embodiments, one or more portions of the transducer surface 2a are bonded to the mount 1 with the sealing 70. In some embodiments, one or more edges of the substrate 2 and one or more portions of the transducer surface 3a are bonded to the mount 1 with the sealing 70. The sealing 70 provides a liquid-tight seal between the transducer surface 2a and the mount 1. By sealing the transducer surface 2a and isolating it from the non-transducer surface 2b, the nebuliser is configured to nebulise the liquid 4 without the liquid contacting the transducer surface 2a. This protects the transducer surface 2a and the electroacoustic transducer(s) 48, 50 from degrading or fouling as a result of operation of the nebuliser. In some embodiments, the substrate 2 may be bonded to the housing similarly to as is described with reference to the substrate 2 being bonded to the mount 1.

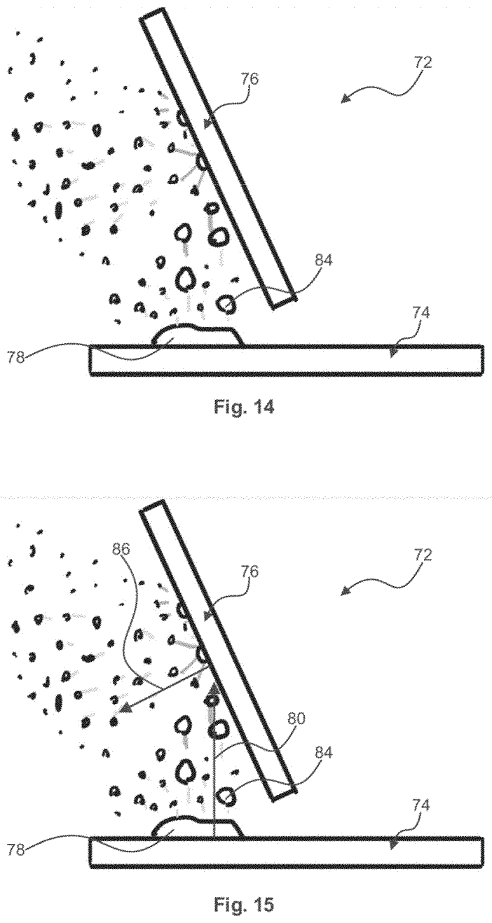

FIGS. 14 and 15 show an embodiment of a nebulising system 72 according to some embodiments. The nebulising system 72 comprises a first nebuliser 74. The first nebuliser 74 may be in the form of any one of the nebulisers described herein. Alternatively, the first nebuliser 74 may be in another form. The nebulising system 74 also comprises a second nebuliser 76. The second nebuliser 76 may be in the form of any one of the nebulisers described herein. Alternatively, the second nebuliser 76 may be in another form. The second nebuliser 76 may be considered an active baffle. The second nebuliser 76 is arranged such that it is angled with respect to the first nebuliser 74. Specifically, the second nebuliser 76 is arranged such that it is transverse to the first nebuliser 74. In other words, a first line that is tangential to a water contacting surface of the first nebuliser 74 (e.g. the transducer surface or the non-transducer surface of the first nebuliser 74) is transverse to a second line that is tangential to a water contacting surface of the second nebuliser 76 (e.g. the transducer surface or the non-transducer surface of the second nebuliser 76). The water contacting surface of the first nebuliser 74 may be a transducer surface (comprising an electroacoustic transducer as previously described) and/or a non-transducer surface of the first nebuliser 74. Similarly, the water contacting surface of the second nebuliser 76 may be a transducer surface (comprising an electroacoustic transducer as previously described) and/or a non-transducer surface of the second nebuliser 76.

Liquid 4 is administered to the first nebuliser 74 at a liquid administration point 78. The first nebuliser 74 nebulises the liquid. A first portion 82 of liquid ejected from the first nebuliser 74 is in the form of relatively small liquid droplets with a diameter less than 3 μm. These relatively small liquid droplets carry little momentum and do not travel far from the first nebuliser 74. A second portion 84 of the liquid ejected from the first nebuliser 74 is in the form of relatively large liquid droplets with a diameter greater than 3 μm. These relatively large liquid droplets carry relatively more momentum that the small liquid droplets with a diameter less than 3 μm and can contact the second nebuliser 76.

Liquid ejected from the first nebuliser 74 may have a first trajectory 80. The first trajectory 80 may, for example, be a generally upwards trajectory. Upon contacting the second nebuliser 76, the liquid droplets are split into smaller droplets (e.g. with a diameter less than 3 μm). Therefore, a significant portion (e.g. a majority or all) of the liquid droplets produced by the nebulising system 72 are of a size below a size threshold. For example, the liquid droplets produced by the nebulising system 72 are of a diameter below a diameter threshold, such as 3 μm. The liquid droplets experience minimal residence time on the second nebuliser 76. The liquid droplets that contact the second nebuliser 76 are directed away from the second nebuliser along a second trajectory 86. The second trajectory 86 is generally transverse to the first trajectory 80. Thus, the second nebuliser 76 may be configured to redirect a portion of liquid nebulised by the first nebuliser 74. Furthermore, the second nebuliser 76 may be considered to nebulise already-nebulised liquid.

As the second nebuliser 76 is angled with respect to the first nebuliser 74, the liquid that contacts the second nebuliser 76 is directed away from the first nebuliser 74 (i.e. the first trajectory 80 and the second trajectory 86 are different). This helps reduce the extent to which the liquid that contacts the second nebuliser 76 recirculates back to the first nebuliser 74.

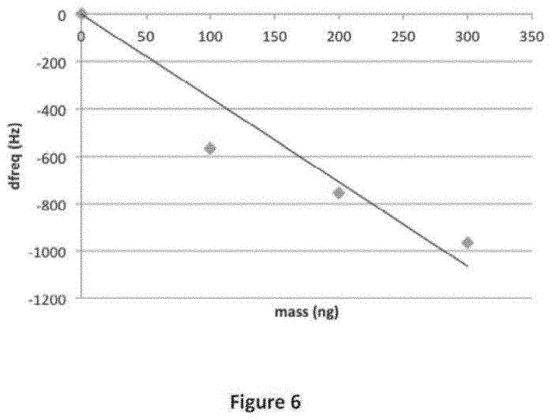

For sensing, the optically flat single crystal substrate allows for bulk (e.g. Lamb) wave resonances that have large quality factors Q in the order of 10⁴ to 10⁶. Therefore, very small mass loadings on the surface of the substrate can produce detectable frequency shifts so as to allow mass sensing of samples down to 10 ng sensitivity. This is shown in the graph of FIG. 6 which shows the mass sensing of Humalog (insulin medication). The graph shows a linear frequency shift with increasing mass, with the sensitivity of 100 ng.

SAW nebulisers have found application in a variety of fields, including in the administration of active agents. Inhaled medication is the most common form of therapy for asthma, chronic obstructive pulmonary disease (COPD) and for other respiratory conditions, such as obstructive bronchitis, emphysema, and cystic fibrosis. For example, corticosteroids, bronchodilators and β2 agonists are typically administered by inhalation for treatment of asthma, COPD and other respiratory conditions. It is envisaged that the described nebuliser may be used in conjunction with a range of possible active agents. Suitable active agents include, but are not limited to, corticosteroids (such as Fluticasone, Budesonide, Mometasone, Beclomethasone, and Ciclesonide), bronchodilators (such as Salmeterol or Albuterol, Formoterol, Vilanterol, Levalbuterol and Ipratropium). By way of example, Albuterol, also referred to as salbutamol or Ventolin, is a β2 agonist and short-term bronchodilator that opens up the medium and large airways in the lungs. Ipratropium, also referred to as Ipratropium bromide, is a muscarinic antagonist (a type of anticholinergic) which opens up the medium and large airways in the lungs. Budesonide, also referred to as BUD, is a type of corticosteroid used for the long-term management of asthma and chronic obstructive pulmonary disease (COPD). In an embodiment, the described nebuliser is adapted for delivery of Albuterol. In an embodiment, the described nebuliser is adapted for delivery of Ipratropium. In an embodiment, the described nebuliser is adapted for delivery of Budesonide.

The described nebuliser advantageously provides reliable, efficient and accurate delivery of active agents. The resultant nebulised liquids may be characterized by one or more parameters. It is appreciated that each active agent has differing physicochemical properties. Furthermore, it is appreciated that various parameters of the described nebuliser may be optimised for delivery of a given active agent, including droplet size (microns), geometric standard deviation (GSD), volumetric atomization rate, stablization period (i.e. time to use), fraction of API administered, trajectory losses, and fine particle fraction.

In an aspect, the described nebuliser provides control of the droplet size of nebulised liquids. In particular, the droplet size of nebulised liquids may be optimised for a given active agent. In an embodiment, the described nebuliser provides nebulised liquids wherein the droplet size is in the range of from 0.1 and 100 μm, preferably in the range of from 0.1 to 10 μm, preferably in the range of from 0.5 to 7.5 μm, more preferably in the range of from 1 to 5 μm, even more preferably in the range of from 2 to 4 μm. In an embodiment, the described nebuliser provides nebulised liquids wherein the droplet size is <10 μm, preferably <8 μm, preferably <6 μm, preferably <5 μm, preferably <3 μm.

In an aspect, the described nebuliser provides control of geometric standard deviation (GSD) of the droplets of nebulised liquids. In particular, the GSD of nebulised liquids may be optimised for a given active agent. In an embodiment, the described nebuliser provides nebulised liquids wherein the GSD is <10 μm, preferably <8 μm, preferably <6 μm, preferably <5 μm, preferably <3 μm, preferably <2.5 μm, preferably <2.1 μm.

In an aspect, the described nebuliser provides control of the stabilization period (i.e. time to use). Advantageously, the described nebuliser provides reduced stabilization periods (i.e. time to use). Short or reduced stabilization periods provide reduced lag-time to use, increased efficiency, reduction in sample loss or fluid loss, and improved accuracy with dosing and administration of active agents. In particular, the stabilization period may be optimised for a given active agent. In an embodiment, the described nebuliser provides a stabilization period of <1 sec, preferably <0.5 sec, preferably <0.25 sec, preferably <0.1 sec, preferably <0.05 sec, preferably <0.03 sec, preferably <0.02 sec, preferably <0.01 sec.

In an aspect, the described nebuliser provides control of the volumetric atomization rate of nebulised liquids. In particular, the volumetric atomization rate of nebulised liquids may be optimised for a given active agent. In an embodiment, the described nebuliser provides nebulised liquids wherein the volumetric atomization rate is in the range of from 0.1 to 10 mL/min, preferably in the range of from 0.15 to 7.5 mL/min, preferably in the range of from 0.2 to 5 mL/min. In an embodiment, the described nebuliser provides nebulised liquids wherein the volumetric atomization rate is >0.1 mL/min, preferably >0.25 mL/min, preferably >0.3 mL/min, preferably >0.35 mL/min, preferably >0.4 mL/min, preferably >0.45 mL/min, preferably >0.5 mL/min, preferably >0.55 mL/min, preferably >0.6 mL/min, preferably >0.65 mL/min, preferably >0.7 mL/min, preferably >0.75 mL/min.

In an aspect, the described nebuliser provides control of the fraction of API administered in nebulised liquids. In particular, the fraction of API administered may depend on the physicochemical properties of a given active agent, but may be optimised for a given active agent with the described system. In an embodiment, the described nebuliser provides nebulised liquids wherein the fraction of API administered is >60%, preferably >65%, preferably >70%, preferably >75%, preferably >80%, preferably >85%, preferably >90%, preferably >95%, preferably >97%, preferably >98%, preferably >99%.

In an aspect, the described nebuliser provides control of the trajectory losses in nebulised liquids. In particular, the trajectory losses may be optimised for a given active agent. In an embodiment, the described nebuliser provides nebulised liquids wherein the trajectory loss is <20%, preferably <15%, preferably <10%, preferably <9%, preferably <8%, preferably <7%, preferably <6%, preferably <5%.

In an aspect, the described nebuliser provides control of the fine particle fraction of nebulised liquids. Fine particle fraction is generally understood as a measure of mass depositing in the lung during inhalation of nearly isotonic nebulised aerosols. The amount of aerosol inhaled in different fine particle definitions is compared to the amount of aerosol depositing in the lung and alveolar regions for nearly isotonic nebulised aerosols. It is accepted that droplet stages 1-7 have 65% drug in a form that accumulates or targets deep lung tissue. The fine particle fraction may depend on the physicochemical properties of a given active agent, but may be optimised for a given active agent with the described system. In an embodiment, the described nebuliser provides a fine particle fraction of >20% in droplet stages 1-7, preferably >30%, preferably >35%, preferably >40%, preferably >45%, preferably >50%, preferably >55%, preferably >60%, preferably >65%, preferably >70%, preferably >75%.

In addition to the active agents described, the described nebuliser may be adapted to nebulise fluids or samples comprising delicate molecules and particles (e.g. DNA, RNAi, peptides, proteins and cells) without denaturing them while maintaining high nebulisation throughout (typically above 1 ml per minute). Prior art nebulisers are to date limited to between 0.1 to 0.4 ml/min thereby necessitating long inhalation times, typically from tens of minutes to an hour. This has therefore limited the practical uptake of conventional nebulisers. The higher nebulisation rates that can be achieved by the nebuliser of the described embodiments can significantly shorten the administration time.

Nebulisers in accordance with the described embodiments have been subject to human clinical trials to determine efficiency of delivery of active agents to the lungs by inhalation using Technetium-99m DTPA aerosol ([^{99m}Tc]DTPA aerosol). Initial results indicate the described nebuliser systems provide effective delivery of nebulised active agent to the target tissue.

TABLE 1
Unadjusted clinical results from initial human
clinical trials with [^{99m}Tc]DTPA aerosol

| | Volunteer 1 | Volunteer 2 | Volunteer 3 | Volunteer 4 |
|---|---|---|---|---|
| Right lung | 8.25 | 27.1 | 21.9 | 26.6 |
| dose (MBq) | | | | |
| Left lung | 7.15 | 25.2 | 22.2 | 23.3 |
| dose (MBq) | | | | |
| Total lung | 15.4 | 52.3 | 44.1 | 49.9 |
| dose(MBq) | | | | |

Modifications and variations as would be deemed obvious to the person skilled in the art are included within the ambit of the present invention as claimed in the appended claims.

In a first aspect, the disclosure provides:
A nebuliser for nebulising liquid droplets, including:
a housing;

at least one piezoelectric substrate accommodated within the housing and having a transducer surface upon which is located at least one electroacoustic transducer for generating acoustic wave energy within the substrate, and an opposing non-transducer surface; and

a liquid supply system for supplying a liquid to at least one of the transducer and non-transducer surfaces, the liquid supply system including a reservoir for accommodating the liquid, and at least one relatively rigid supply conduit in contact with the substrate for supplying the liquid from the reservoir to the substrate.

In a second aspect, the disclosure provides:
A nebuliser for nebulising liquid droplets, including:
a housing;

at least one piezoelectric substrate accommodated within the housing and having a transducer surface upon which is located at least one electroacoustic transducer for generating acoustic wave energy within the substrate, and an opposing non-transducer surface;

a liquid supply system for supplying a liquid to at least one of the transducer and non-transducer surfaces, the liquid supply system including a reservoir for accommodating the liquid, and at least one relatively rigid supply conduit in contact with the substrate for supplying the liquid from the reservoir to the substrate; and
a sensor for detecting the volume of liquid on the surface of the substrate.

In a third aspect, the disclosure provides:
A nebuliser for nebulising liquid droplets, including:
a housing;

at least one piezoelectric substrate accommodated within the housing and having a transducer surface upon which is located at least one electroacoustic transducer for generating acoustic wave energy within the substrate, and an opposing non-transducer surface
a compliant material in contact with at least a portion of the perimeter surface of the at least one piezoelectric substrate; and

a liquid supply system for supplying a liquid to at least one of the transducer and non-transducer surfaces, the liquid supply system including a reservoir for accommodating the liquid, and at least one supply conduit for supplying the liquid from the reservoir to the substrate.

In a fourth aspect, the disclosure provides:
A nebuliser for nebulising liquid droplets, including:
a housing;

at least one piezoelectric substrate accommodated within the housing and having a transducer surface upon which is located at least one electroacoustic transducer for generating acoustic wave energy within the substrate, and an opposing non-transducer surface
a compliant material in contact with at least a portion of the perimeter surface of the at least one piezoelectric substrate;

a liquid supply system for supplying a liquid to at least one of the transducer and non-transducer surfaces, the liquid supply system including a reservoir for accommodating the liquid, and at least one supply conduit for supplying the liquid from the reservoir to the substrate; and
a sensor for detecting the volume of liquid on the surface of the substrate.
   A nebuliser according to the first or second aspect, therein the supply conduit is in the form of a nib or needle.
   A nebuliser according to the first or second aspect, wherein the supply conduit is formed from an acoustically reflecting material.
   A nebuliser according to the first or second aspect, wherein the liquid is gravity fed from the reservoir through the supply conduit.
   A nebuliser according to any one of the aspects, wherein the liquid is transferred from the reservoir through an active pumping system.
   A nebuliser according to any one of the aspects, wherein the active pumping system is a syringe or peristaltic pump.
   A nebuliser according to any one of the aspects, wherein the liquid supply system further includes a flow regulator for providing a steady flow of liquid therefrom.
   A nebuliser according to any one of the aspects, wherein the flow regulator includes a liquid outlet passage through which liquid can pass, and an air inlet passage connected to the reservoir.
   A nebuliser according to any one of the aspects, wherein the sensor detects the volume of liquid on the surface of the substrate by measuring a change in current across the nebuliser system.
   A nebuliser according to any one of the aspects, wherein the current is direct current.
   A nebuliser according to any one of the aspects, wherein the nebuliser system across which change in current is detected includes an electronic circuit and at least one piezoelectric substrate.
   A nebuliser according to any one of the aspects, wherein the electronic circuit includes at least one printed circuit board.
   A nebuliser according to any one of the aspects, further comprising a control switch responsive to the sensor for controlling the operation of the nebuliser.
   A nebuliser according to any one of the aspects, wherein the nebuliser further comprises at least one opposing electroacoustic transducer for generating acoustic wave energy in an opposing direction to prevent liquid from being driven off the surface of the substrate prior to nebulisation.
   A nebuliser according to any one of the aspects, wherein the substrate further comprises a structure for containing and/or preventing loss of liquid applied to the surface prior to nebulisation.

A nebuliser according to any one of the aspects, wherein the structure comprises a lip, a wall, a gasket, a deposited raised film or combinations thereof.
A nebuliser according to any one of the aspects, further including an inner chamber connected to the flow regulator, the inner chamber having a peripheral opening within which is accommodated a peripheral tip of the supply conduit, wherein liquid can pass through capillary action between the peripheral opening and the peripheral tip of the supply conduit.
A nebuliser according to any one of the aspects, wherein the substrate is supported on a displaceable mount for controlling the contact of the substrate with the supply conduit.
A nebuliser according to any one of the aspects, wherein the mount includes a pivot support at one end thereof and an opposing end supported on a resilient member.
A nebuliser according to any one of the aspects, wherein the mount is supported on a cantilever.
A nebuliser according to any one of the aspects, further including a control means for controlling the size of the nebulised liquid droplets.
A nebuliser according to any one of the aspects, wherein the control means includes at least one baffle located in a generally parallel and adjacent relationship to at least one of transducer surfaces.
A nebuliser according to any one of the aspects, wherein the baffle is provided by a housing inner wall located in a parallel adjacent relationship from at least one said substrate surface.
A nebuliser according to any one of the aspects, wherein the housing further includes an inlet opening, and the reservoir includes a neck portion that can be accommodated within the inlet opening.
A nebuliser according to any one of the aspects, including at least two said substrates spaced apart and located in a parallel adjacent relationship.
A nebuliser according to any one of the aspects, wherein the droplet size control means includes pre-setting the spacing between the substrates to control the thickness of the meniscus of the liquid supplied between the adjacent substrate surfaces, to thereby control the size of the nebulised droplets.
A nebuliser according to any one of the aspects, wherein the droplet size control means includes pre-setting the spacing of the substrates from internal walls of the housing to control the thickness of the meniscus of the liquid supplied between the adjacent substrate surface and inner wall, to thereby control the size of the nebulised droplets.
A nebuliser according to any one of the aspects wherein the droplet size control means includes a liquid film forming structure at the interface between the liquid supply conduit and the substrate to control the thickness of the meniscus of the liquid supplied between the adjacent substrate surface to thereby control the size of the nebulised droplets.
A nebuliser according to any one of the aspects, wherein the liquid film forming structure includes a web, mesh, one or more fibres, or a slot in the liquid supply conduit
A nebuliser according to any one of the aspects, wherein the piezoelectric substrate and electroacoustic transducer is also used to sense a liquid mass on the at least one substrate.
A nebuliser according to any one of the aspects, wherein the compliant material is selected from is selected from the group consisting of adhesive tape, silicone rubber and thermal paste, or combinations thereof.
A nebuliser according to any one of the aspects, wherein the compliant material is in contact with at least a portion of the perimeter of the distal end of substrate.
A nebuliser according to the third or fourth aspects, wherein the at least one supply conduit is a relatively rigid supply conduit in contact with the substrate.
A nebuliser according to the third or fourth aspects, wherein the at least one supply conduit is selected from the group consisting of a nib, a needle, a wick, a microchannel, or combinations thereof.
A nebuliser according to any one of the aspects, wherein at least of portion of the transducer surface, the non-transducer surface, or combinations thereof is patterned.
A nebuliser according to any one of the aspects, wherein the acoustic wave energy includes surface acoustic waves (SAW) propagated in the transducer surface of the at least one substrate.
A nebuliser according to any one of the aspects, wherein the acoustic wave energy includes surface reflected bulk waves (SRBW) reflected between the transducer and non-transducer surfaces of the at least one substrate.
A nebuliser according to any one of the aspects, wherein the acoustic wave energy includes a combination of surface acoustic waves (SAW) propagated in the transducer surface of the at least one substrate and surface reflected bulk waves (SRBW) reflected between the transducer and non-transducer surfaces of the at least one substrate.
A nebuliser according to any one of the aspects, wherein the surface acoustic waves (SAW) include standing waves, traveling waves and combinations thereof.
A nebuliser according to any one of the aspects, wherein the surface reflected bulk waves (SRBW) include standing waves, traveling waves and combinations thereof.
A nebuliser according to any one of the aspects, wherein the electroacoustic transducer is an interdigital transducer (IDT).
A nebuliser according to any one of the aspects, wherein the at least one piezoelectric substrate has a thickness at or around a wavelength of the SAW propagated in the transducer surface.
A nebuliser according to any one of the aspects, wherein the at least one piezoelectric substrate is formed of Lithium Niobate (LiNbO₃).
A nebuliser according to any one of the aspects, wherein at least a portion of the non-transducer surface further includes a coating comprising at least one metal.
A nebuliser according to any one of the aspects, wherein at least a portion of the transducer surface further includes a coating at the distal end of the substrate comprising at least one metal.
A nebuliser according to any one of the aspects wherein the coating comprises, titanium, gold, aluminium, chromium or combinations thereof.
A nebuliser according to any one of the aspects, wherein the liquid is nebulised from the transducer surface, the non-transducer surface, or both the transducer surface and the non-transducer surface.
A nebuliser according to any one of the aspects, wherein the liquid is nebulised to form droplets having a size range between 0.1 and 100 μm.
A nebuliser according to any one of the aspects, wherein the liquid is nebulised at a nebulisation rate of up to 10 ml/min.
A nebuliser according to any one of the aspects, wherein the mount includes a shelf upon which the substrate is mounted, the shelf including one or more gaps for preventing liquid creep along the substrate.
A nebuliser according to any one of the aspects, wherein the housing is in the form of a cartridge housing having an external electrical contact connected to the at least one electroacoustic transducer, and an integral liquid supply system.
A method of nebulising a liquid using a nebuliser according to the first or second aspects.
A method of nebulising according to any one of the aspects, including nebulising liquid to form liquid droplets having a size range between 0.1 and 100 μm.
A method of nebulising to any one of the aspects, including nebulising liquid at a volumetric nebulisation rate of up to 10 ml/min.
A method of nebulising according to any one of the aspects, including nebulising liquid to form liquid droplets having geometric standard deviation (GSD) of <10 μm.
A method of nebulising according to any one of the aspects, wherein the liquid includes functional or therapeutic agents therein such as pharmaceuticals, DNA, RNAi, peptides, proteins and cells, or, non-therapeutic agents such as perfume, cosmetics, pesticides, paints or antiseptics.
A method of nebulising according to any one of the aspects, wherein the functional or therapeutic agent is delivered as a unit dose.
A method of nebulising according to any one of the aspects, wherein the unit dose is determined by a sensor for detecting the volume of liquid on the surface of the substrate.

1. A nebuliser for nebulising liquid droplets, including:
a housing;
at least one piezoelectric substrate accommodated within the housing and having a transducer surface upon which is located at least one electroacoustic transducer for generating acoustic wave energy within the at least one piezoelectric substrate, and an opposing non-transducer surface;
a liquid supply system for supplying a liquid to at least one of the transducer and non-transducer surfaces, the liquid supply system including a reservoir for accommodating the liquid, and at least one relatively rigid supply conduit in contact with the at least one piezoelectric substrate for supplying the liquid from the reservoir to the at least one piezoelectric substrate; and
a sensor for detecting a volume of liquid on the at least one piezoelectric substrate.

2. The nebuliser according to claim 1, therein the supply conduit is in the form of a nib or needle.

3. A nebuliser for nebulising liquid droplets, including:
a housing;
at least one piezoelectric substrate accommodated within the housing and having a transducer surface upon which is located at least one electroacoustic transducer for generating acoustic wave energy within the at least one piezoelectric substrate, and an opposing non-transducer surface;
a compliant material in contact with at least a portion of a perimeter surface of the at least one piezoelectric substrate;
a liquid supply system for supplying a liquid to at least one of the transducer and non-transducer surfaces, the liquid supply system including a reservoir for accommodating the liquid, and at least one supply conduit for supplying the liquid from the reservoir to the at least one piezoelectric substrate; and
a sensor for detecting a volume of liquid on the at least one piezoelectric substrate.

4. The nebuliser according to claim 3, wherein the compliant material is selected from the group consisting of: adhesive tape, silicone rubber, thermal paste, or combinations thereof.

5. The nebuliser according to claim 3 or 4, wherein the compliant material is in contact with at least a portion of a perimeter of a distal end of at least one the piezoelectric substrate.

6. The nebuliser according to any one of claims 3 to 5, wherein the at least one supply conduit is a relatively rigid supply conduit in contact with the at least one piezoelectric substrate.

7. The nebuliser according to any one of claims 3 to 5, wherein the at least one supply conduit is selected from the group consisting of a nib, a needle, a wick, a microchannel, or combinations thereof

8. The nebuliser according to any one of the preceding claims, wherein the sensor detects the volume of liquid on the surface of the at least one piezoelectric substrate by measuring a change in current across the nebuliser.

9. The nebuliser according to claim 8, wherein the current is direct current.

10. The nebuliser according to any one of the preceding claims, wherein the sensor is configured to detect a volume of liquid on the transducer surface and/or the non-transducer surface of the at least one piezoelectric substrate.

11. The nebuliser according to claim 10, wherein the electronic circuit includes at least one printed circuit board.

12. The nebuliser according to any one of the preceding claims further comprising a control switch responsive to the sensor for controlling operation of the nebuliser.

13. The nebuliser according to any one of the preceding claims wherein the nebuliser further comprises at least one opposing electroacoustic transducer for generating acoustic wave energy in an opposing direction to reduce an extent to which liquid is driven off the at least one piezoelectric substrate prior to nebulisation.

14. The nebuliser according to any one of the preceding claims wherein the at least one piezoelectric substrate further comprises a containing barrier structure for containing and/or preventing loss of liquid applied to the piezoelectric substrate prior to nebulisation.

15. The nebuliser according to claim 14 wherein the containing barrier structure comprises a lip, a wall, a gasket, a deposited raised film, and combinations thereof.

16. The nebuliser according to any one of the preceding claims, wherein the liquid is i) gravity fed from the reservoir, or ii) transferred from the reservoir via an active pumping system.

17. The nebuliser according to any one of the preceding claims, wherein the liquid supply system further includes a flow regulator for providing a steady flow of liquid therefrom.

18. The nebuliser according to any one of the preceding claims, wherein the at least one piezoelectric substrate is supported on a displaceable mount for controlling the contact of the at least one piezoelectric substrate with the supply conduit.

19. The nebuliser according to any one of the preceding claims, further including a control means for controlling a size of the nebulised liquid droplets.

20. The nebuliser according to claim 19, wherein the control means includes at least one baffle located in a generally parallel and adjacent relationship to at least one of the transducer surface or the non-transducer surface.

21. The nebuliser according to claim 20, wherein the baffle is provided by a housing inner wall located in a parallel adjacent relationship with respect to at least one of the transducer surface or the non-transducer surface.

22. The nebuliser according to any one of the preceding claims, wherein the housing further includes an inlet opening, and the reservoir includes a neck portion that can be accommodated within the inlet opening.

23. The nebuliser according to any one of the preceding claims, including at least two piezoelectric substrates spaced apart and located in a parallel adjacent relationship.

24. The nebuliser according to claim 23 when dependent on claim 19 or any one of claims 20 to 22 when dependent on claim 19, wherein the droplet size control means is configured to enable pre-setting of a spacing between the at least two piezoelectric substrates to control a thickness of a meniscus of liquid supplied between adjacent substrate surfaces, to thereby control the size of the nebulised droplets.

25. The nebuliser according to claim 23 when dependent on claim 21, wherein the droplet size control means is configured to enable pre-setting of a spacing of the at least two piezoelectric substrates from internal walls of the housing to control a thickness of a meniscus of liquid supplied between adjacent substrate surfaces and the inner walls, to thereby control the size of the nebulised droplets.

26. The nebuliser according to claim 19, wherein the droplet size control means includes a liquid film forming structure in fluid communication with the liquid supply conduit and the at least one piezoelectric substrate to control a thickness of a meniscus of liquid supplied to the at least one piezoelectric substrate to thereby control the size of the nebulised droplets.

27. The nebuliser according to claim 26, wherein the liquid film forming structure includes a web, mesh, one or more fibres, or a slot in the liquid supply conduit.

28. The nebuliser according to any one of the preceding claims, wherein at least of portion of the transducer surface, the non-transducer surface, or combinations thereof is patterned.

29. The nebuliser according to any one of the preceding claims, wherein the acoustic wave energy includes surface acoustic waves (SAW) propagated in the transducer surface of the at least one piezoelectric substrate.

30. The nebuliser according to any one of the preceding claims, wherein the acoustic wave energy includes surface reflected bulk waves (SRBW) reflected between the transducer and non-transducer surfaces of the at least one piezoelectric substrate.

31. The nebuliser according to any one of claims 1 to 28, wherein the acoustic wave energy includes a combination of surface acoustic waves (SAW) propagated in the transducer surface of the at least one piezoelectric substrate and surface reflected bulk waves (SRBW) reflected between the transducer and non-transducer surfaces of the at least one piezoelectric substrate.

32. The nebuliser according to claim 29 or 31, wherein the surface acoustic waves (SAW) include standing waves, traveling waves and combinations thereof.

33. The nebuliser according to claim 30 or 31, wherein the surface reflected bulk waves (SRBW) include standing waves, traveling waves and combinations thereof.

34. The nebuliser according to any one of the preceding claims, wherein the electroacoustic transducer is an interdigital transducer (IDT).

35. The nebuliser according to any one of claims 29 to 34, wherein the at least one piezoelectric substrate has a thickness at or around a wavelength of the SAW propagated in the transducer surface.

36. The nebuliser according to any one of the preceding claims, wherein the at least one piezoelectric substrate is formed of Lithium Niobate (LiNbO₃).

37. The nebuliser according to any one of the preceding claims, wherein the liquid is nebulised from the transducer surface, the non-transducer surface, or both the transducer surface and the non-transducer surface.

38. The nebuliser according to any one of the preceding claims, wherein the liquid is nebulised to form droplets having a size range between 0.1 and 100 μm.

39. The nebuliser according to any one of the preceding claims, wherein the liquid is nebulised at a nebulisation rate of up to 10 ml/min.

40. The nebuliser according to any one of the preceding claims, wherein the housing is in the form of a cartridge housing having an external electrical contact connected to the at least one electroacoustic transducer, and an integral liquid supply system.

41. The nebuliser according to claim 18 or any one of claims 19 to 40 when dependent on claim 18, the at least one piezoelectric substrate is bonded to the displaceable mount.

42. The nebuliser according to claim 41, wherein the at least one piezoelectric substrate is bonded to the displaceable mount with a sealing that provides a liquid-tight seal between the transducer surface and the displaceable mount.

43. The nebuliser according to any one of claims 1 to 40, wherein the at least one piezoelectric substrate is bonded to the housing with a sealing that provides a liquid-tight seal between the transducer surface and the housing.

44. The nebuliser according to any one of claims 1 to 43, wherein the non-transducer surface comprises one or more electroacoustic transducers.

45. A nebuliser for nebulising liquid droplets, including:
a housing;
at least one piezoelectric substrate accommodated within the housing and having a transducer surface upon which is located at least one electroacoustic transducer for generating acoustic wave energy within the at least one piezoelectric substrate;
at least one opposing electroacoustic transducer for generating acoustic wave energy in an opposing direction to reduce an extent to which liquid is driven off the transducer surface prior to nebulisation; and
a liquid supply system for supplying a liquid to the at least one piezoelectric substrate.

46. A nebuliser for nebulising liquid droplets, including:
a housing;
at least two piezoelectric substrates accommodated within the housing; each having a respective transducer surface upon which is located at least one electroacoustic transducer for generating acoustic wave energy within the respective piezoelectric substrate;
wherein the at least two piezoelectric substrates are spaced apart and located in a parallel adjacent relationship;
a liquid supply system for supplying a liquid to at least one of the piezoelectric substrates; and
a control means for controlling a size of the nebulised liquid droplets, the control means being configured to enable pre-setting of a spacing between the at least two piezoelectric substrates to control a thickness of a meniscus of liquid supplied between adjacent substrate surfaces, to thereby control the size of the nebulised droplets.

47. A nebuliser for nebulising liquid droplets, including:
a housing;
at least one piezoelectric substrate accommodated within the housing and having a transducer surface upon which is located at least one electroacoustic transducer for generating acoustic wave energy within the substrate, and an opposing non-transducer surface; and
a liquid supply system for supplying a liquid to at least one of the transducer and non-transducer surfaces, the liquid supply system including a reservoir for accommodating the liquid, and at least one relatively rigid supply conduit in contact with the at least one piezoelectric substrate for supplying the liquid from the reservoir to the at least one piezoelectric substrate.

48. A nebuliser for nebulising liquid droplets, including:
a housing;
at least one piezoelectric substrate accommodated within the housing and having a transducer surface upon which is located at least one electroacoustic transducer for generating acoustic wave energy within the at least one piezoelectric substrate, and an opposing non-transducer surface;
a compliant material in contact with at least a portion of a perimeter surface of the at least one piezoelectric substrate; and
a liquid supply system for supplying a liquid to at least one of the transducer and non-transducer surfaces, the liquid supply system including a reservoir for accommodating the liquid, and at least one supply conduit for supplying the liquid from the reservoir to the at least one piezoelectric substrate.

49. The nebuliser according to any one of claims 1 to 48, wherein the at least one electroacoustic transducer is configured to provide an output indicative of a volume of liquid on the at least one piezoelectric substrate.

50. The nebuliser according to claim 49, wherein the output provided by the at least one electroacoustic transducer is a current.

51. The nebuliser according to any one of claims 45 to 48, further comprising a sensor for detecting a volume of liquid on the at least one piezoelectric substrate.

52. The nebuliser according to claim 51, wherein the at least one electroacoustic transducer comprises the sensor.

53. The nebuliser according to any one of claims 45 to 52, further comprising at least one opposing electroacoustic transducer for generating acoustic wave energy in an opposing direction to reduce an extent to which liquid is driven off the at least one piezoelectric substrate prior to nebulisation.

54. The nebuliser according to claim 53, wherein the at least one opposing electroacoustic transducer is configured to provide an output indicative of a volume of liquid on the at least one piezoelectric substrate.

55. The nebuliser according to claim 54, wherein the output provided by the at least one opposing electroacoustic transducer is a current.

56. The nebuliser according to claim 53 when dependent on claim 51, wherein the at least one opposing electroacoustic transducer comprises the sensor.

57. The nebuliser according to any one of claims 45 to 56, further including a control means for controlling a size of the nebulised liquid droplets.

58. The nebuliser according to any one of claims 45 to 57, including at least two piezoelectric substrates spaced apart and located in a parallel adjacent relationship.

59. The nebuliser according to claim 58 when dependent on claim 57, wherein the droplet size controlling means is configured to enable pre-setting of a spacing between the at least two piezoelectric substrates to control a thickness of a meniscus of liquid supplied between adjacent substrate surfaces, to thereby control the size of the nebulised droplets.

60. The nebuliser according to claim 58 when dependent on claim 57, wherein the droplet size controlling means is configured to enable pre-setting of a spacing of the at least two piezoelectric substrates from internal walls of the housing to control a thickness of a meniscus of liquid supplied between adjacent substrate surfaces and the inner walls, to thereby control the size of the nebulised droplets.

61. The nebuliser according to claim 57, wherein the droplet size control means includes a liquid film forming structure in fluid communication with the liquid supply conduit and the at least one piezoelectric substrate to control a thickness of a meniscus of liquid supplied to the at least one piezoelectric substrate to thereby control the size of the nebulised droplets.

62. The nebuliser according to claim 61, wherein the liquid film forming structure includes a web, mesh, one or more fibres, or a slot in the liquid supply conduit.

63. A nebuliser system comprising:
a nebuliser according to any one of the preceding claims, wherein the nebuliser is a first nebuliser; and
a second nebuliser.

64. The nebuliser system of claim 63, wherein:
the first nebuliser comprises a first nebuliser water contacting surface;
the second nebuliser comprises a second nebuliser water contacting surface.

65. The nebuliser system of claim 64, wherein the first nebuliser water contacting surface is transverse to the second nebuliser water contacting surface.

66. The nebuliser system of claim 64 or claim 65, wherein:
the first nebuliser water contacting surface is the transducer surface or the non-transducer surface; and
the second nebuliser water contacting surface is a transducer surface of the second nebuliser or a non-transducer surface of the second nebuliser.

67. A method of nebulising a liquid using a nebuliser according to any one of claims 1 to 62 or the nebuliser system of any one of claims 63 to 66.

68. The method of nebulising a liquid according to claim 67, including nebulising liquid to form liquid droplets having a size range between 0.1 and 100 μm.

69. The method of nebulising a liquid according to claim 67 or claim 68, including nebulising liquid at a volumetric nebulisation rate of up to 10 ml/min.

70. The method of nebulising a liquid according to any one of claims 67 to 69, including nebulising liquid to form liquid droplets having geometric standard deviation (GSD) of <10 μm.

71. The method of nebulising a liquid according to any one of claims 6777 to 70, wherein the liquid includes functional or therapeutic agents therein such as pharmaceuticals, DNA, RNAi, peptides, proteins and cells, or, non-therapeutic agents such as perfume, cosmetics, pesticides, paints or antiseptics.

72. The method according to claim 71 wherein the functional or therapeutic agent is delivered as a unit dose.

73. The method according to claim 72 wherein the unit dose is determined by a sensor for detecting the volume of liquid on the at least one substrate.
